(19) United States
(12) Patent Application Publication
(10) Pub. No.: US 2019/0337978 A1
(43) Pub. Date: Nov. 7, 2019

(54) CRYSTALLIZATION METHODS USING FUNCTIONALIZED NANOPOROUS MATRICES AND RELATED SYSTEMS

(71) Applicant: Massachusetts Institute of Technology, Cambridge, MA (US)

(72) Inventors: Allan S. Myerson, Boston, MA (US); Leia Dwyer, Providence, RI (US)

(73) Assignee: Massachusetts Institute of Technology, Cambridge, MA (US)

(21) Appl. No.: 16/402,424

(22) Filed: May 3, 2019

Related U.S. Application Data
(60) Provisional application No. 62/666,250, filed on May 3, 2018.

(51) Int. Cl. C07K 1/30; C07C 213/10; C07C 67/52; C07D 213/56

(52) U.S. Cl. CPC C07K 1/306; C07C 213/10; C07C 2601/16; C07D 213/56; C07C 67/52

(57) ABSTRACT

Methods of forming crystals including, for example, methods of forming crystals using a nanoporous matrix, and associated materials, articles, and systems are generally provided. Advantageously, in some embodiments, the methods of forming crystals described herein provide crystal formation of a target molecule from a solution initially at an undersaturated state with respect to the target molecule. In some embodiments, a method involves obtaining a solution of a target molecule, where in some cases the solution is at an undersaturated state with respect to the target molecule; and exposing the solution to a functionalized nanoporous matrix under conditions that promote crystal formation of the target molecule. In some cases, the nanoporous matrix is functionalized with an antisolvent group.

### RELATED APPLICATIONS

This application claims priority under 35 U.S.C. § 119(e) to U.S. Provisional Patent Application Ser. No. 62/666,250, filed May 3, 2018, and entitled “CRYSTALLIZATION METHODS USING FUNCTIONALIZED NANOPOROUS MATRICES AND RELATED SYSTEMS,” which is incorporated herein by reference in its entirety for all purposes.

### GOVERNMENT SPONSORSHIP

This invention was made with Government support under Grant No. W911NF-16-2-0023 awarded by the Army Research Office. The Government has certain rights in the invention.

### FIELD

The present disclosure generally relates to methods of forming crystals including, for example, methods of forming crystals using a functionalized nanoporous matrix, and associated materials, articles, and systems.

### BACKGROUND

Crystallization is an important type of separation and purification technique, especially in the pharmaceutical industry. Due at least to strict regulations on product quality including crystal form, particle size distribution, crystal shape, purity, and yield, finely controlling crystallization processes for pharmaceuticals and other target molecules is an important goal. Crystallization processes include both nucleation and growth of one or more crystals, both of which stages have implications for an end product of a crystallization process. Nucleation can occur in a solution in a supersaturated state, in which a concentration of a solute in solution is greater than an equilibrium concentration of solute in solution at conditions of temperature, pressure, and/or compositions etc. to which the solution is exposed. Generally, a supersaturated state has been generated in a solution by changing solvent(s) with addition of one or more antisolvents, or by cooling the solution, both of which methods present challenges.

Accordingly, improved methods and systems are needed.

### SUMMARY

The present disclosure generally relates to methods of forming crystals including, for example, methods of forming crystals using a nanoporous matrix, and associated materials, articles, and systems.

Methods are provided for forming crystals. In one aspect, a method of forming crystals comprises: obtaining a solution of a target molecule, wherein the solution is at an undersaturated state with respect to the target molecule; and exposing the solution to a functionalized nanoporous matrix under conditions that promote crystal formation of the target molecule.

In another aspect, a method of forming crystals comprises: obtaining a solution of a target molecule; and exposing the solution to an antisolvent-functionalized nanoporous matrix, under conditions that promote crystal formation of the target molecule.

Systems are provided for forming crystals. In one aspect, a system for forming crystals comprises: a first container comprising a functionalized nanoporous matrix; and a second container comprising a solution of a target molecule, wherein the solution is undersaturated with respect to the target molecule.

In another aspect, a system for forming crystals comprises: a container comprising an antisolvent-functionalized nanoporous matrix; and a solution of a target molecule, wherein the antisolvent-functionalized nanoporous matrix is configured and arranged to promote crystallization of the target molecule from the solution.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 is a non-limiting flow chart illustrating methods 100 of forming crystals of a target molecule;

FIG. 2 is a non-limiting flow chart illustrating methods 200 of forming crystals of a target molecule;

FIG. 3 is a non-limiting schematic diagram of an apparatus comprising a porous matrix material in a glass column through which a solution comprising a target molecule was pumped;

FIG. 4 is a non-limiting plot of differential scanning calorimetry (DSC) scans of samples loaded onto a porous glass matrix from a 550 mg/mL solution of target molecule fenofibrate (FEN) in ethyl acetate solvent;

FIG. 5 is a non-limiting plot of average FEN loading on functionalized Zorbax® chromatographic media (e.g. matrix) versus parent solution concentration of FEN in ethyl acetate solvent;

FIG. 6A and FIG. 6B are non-limiting plots of flowthrough concentration of diphenhydramine hydrochloride (DPH) collected as a control column was run with un-functionalized controlled pore glass (CPG), with corresponding experimental results with Zorbax® chromatographic media in FIG. 8A and FIG. 8B;

FIG. 7 is a non-limiting plot of DSC scans for DPH with bulk melting temperature only seen in control CPG powder (e.g. matrix) collected from column runs;

FIG. 8A and FIG. 8B are non-limiting plots of flowthrough concentration of DPH in isopropyl alcohol on runs (e.g. methods) where a column was packed with Zorbax®;

FIG. 9 is a non-limiting plot of DSC scans of Zorbax® material collected from a column run forming crystals of DPH; and

FIG. 10A-FIG. 10D are non-limiting plots of time series x-ray powder diffraction (XRPD) scans of active pharmaceutical ingredient (API) solutions loaded in capillary tubes filled with Zorbax® media;

FIG. 11 shows non-limiting XRPD scans for capillaries containing Zorbax and diphenhydramine hydrochloride systems;

FIG. 12 shows non-limiting XRPD scans for diphenhydramine systems;

FIG. 13 shows non-limiting XRPD scans for Aspirin systems;

FIG. 14 shows non-limiting XRPD scans for Nicotinamide systems; and

FIG. 15 shows a non-limiting DSC scan for functionalized Zorbax.

### DETAILED DESCRIPTION

Methods and systems for forming crystals including, for example, methods of forming crystals using a nanoporous matrix, and associated materials, articles, and systems are generally provided. Advantageously, in some embodiments, the methods of forming crystals described herein provide crystal formation of a target molecule from a solution initially at an undersaturated state with respect to the target molecule. In some embodiments, a method involves obtaining a solution of a target molecule, where in some cases the solution is at an undersaturated state with respect to the target molecule; and exposing the solution to a functionalized nanoporous matrix under conditions that promote crystal formation of the target molecule. In some cases, the nanoporous matrix is functionalized with an antisolvent group.

Relative to crystallization methods involving an antisolvent, some methods described herein advantageously do not consume antisolvent, but instead involve one or more antisolvent groups functionalizing a surface of a nanoporous matrix, which antisolvent-functionalized nanoporous matrix may be reused a plurality of times without consuming antisolvent groups. As non-limiting examples, antisolvent groups may be attached to a surface of a nanoporous matrix by covalent bonds or metallic bonds or another intermolecular interaction that is not removed in the presence of a solution from which a target molecule is crystallized.

Some methods described herein are preferable to cooling crystallization methods, e.g., in cases where a target molecule does not have a sufficient change in solubility in a solution as a function of temperature and therefore cooling alone would not result in crystal formation of the target molecule, but rather for example the solvent would freeze before crystal formation could occur. Some methods described herein would allow for crystallization of such a target molecule in such a solution. In some embodiments, an antisolvent-functionalized nanoporous matrix, with one or more antisolvent groups on one or more surfaces of the nanoporous matrix (e.g., within one or more nanopores), provides for locally reduced solubility of a target molecule in a solution, with the solubility locally reduced proximate to the one or more antisolvent groups. Without wishing to be bound by theory, this locally reduced solubility may occur especially within the one or more nanopores due to a confinement effect. In some embodiments, such locally reduced solubility results in crystal formation of the target molecule within one or more nanopores.

Some methods described herein may be applied as purification methods. For example, in some embodiments, a target molecule changes in solubility in a solution within a functionalized nanoporous matrix by an amount sufficient to form one or more crystals of the target molecule, whereas an impurity in the solution does not precipitate from the solution. In some embodiments, upon flowing the solution through at least a portion of the nanoporous matrix, impurities are removed and one or more crystals of the target molecule are left behind. In some embodiments, upon subsequently eluting the one or more crystals of the target molecule from the nanoporous matrix using one or more solvents absent impurities, a solution of the target molecule absent impurities results.

Methods and Systems for Forming Crystals

In some embodiments, methods are provided. In some embodiments, methods of forming crystals (e.g., crystals comprising a target molecule) are provided. In some embodiments, one or more crystals result from a method described herein, the one or more crystals having an average largest dimension of between or equal to 0.5 nm and 1 micron. In some embodiments, one or more crystals result from a method described herein, the one or more crystals having an largest dimension of between or equal to 0.5 nm and 100 nm. In some embodiments, one or more crystals result from a method described herein, the one or more crystals having an average largest dimension of between or equal to 0.5 nm and 20 nm.

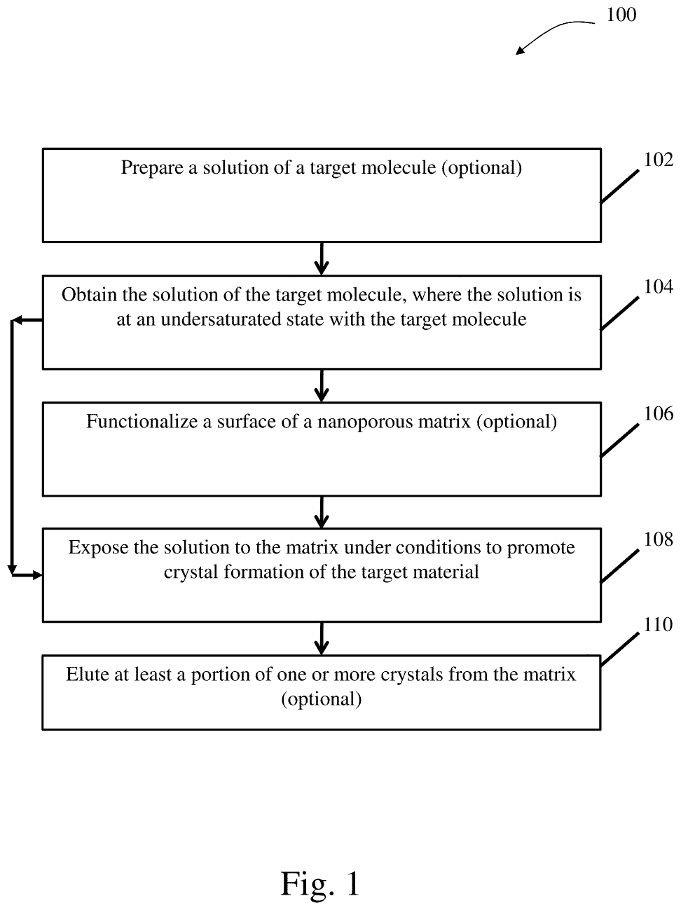

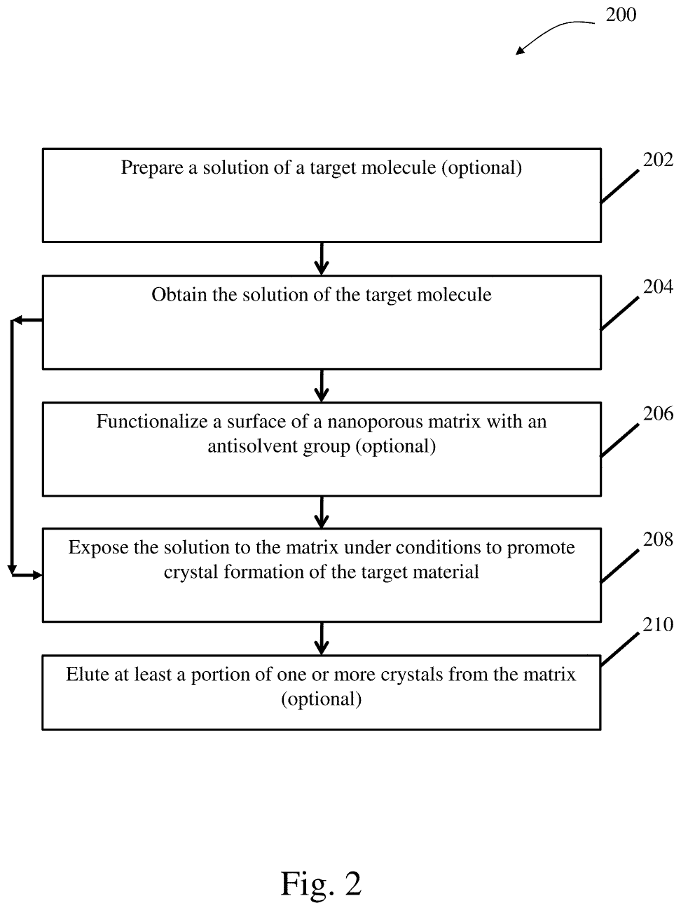

In some embodiments, a method comprises obtaining a solution of a target molecule (e.g., step 104 of FIG. 1, step 204 of FIG. 2). In some embodiments, a solution of a target molecule comprises the target molecule, as is further described herein. In some embodiments, a solution of a target molecule comprises a solvent and one or more additional components. In some embodiments, the solution is at an undersaturated state with respect to the target molecule, as is further described herein.

In some embodiments, a method comprises exposing a solution to a functionalized nanoporous matrix (e.g., step 108 of FIG. 1, step 208 of FIG. 2). In some embodiments, a method involves exposing a solution to a matrix under conditions that promote crystal formation of a target molecule within at least one nanopore in the matrix. In some embodiments, a method involves exposing a solution, which solution is at an undersaturated state with respect to a target molecule, to a matrix under conditions such that the state of the solution is changed to a saturated or supersaturated state with respect to a target molecule. In some embodiments, this change in state of the solution drives crystal formation of the target molecule.

In some embodiments, exposing comprises flowing a solution through at least a portion of a matrix. In some embodiments, flowing comprises pumping a solution through at least a portion of a matrix using a pump. In some embodiments, exposing comprises flowing a solution through at least a portion of the matrix at a constant rate.

In some embodiments, a method comprises exposing a solution to a functionalized nanoporous matrix under conditions that promote crystal formation of the target molecule. In some embodiments, conditions that promote crystal formation of the target molecule include isothermal conditions. In some embodiments, conditions that promote crystal formation of the target molecule include a temperature of an environment external to the solution and the matrix, of between or equal to a melting temperature and a boiling temperature of the solution at 1 atm pressure. In some embodiments, conditions that promote crystal formation of the target molecule include a temperature of an environment external to the solution and the matrix, of between or equal to a melting temperature and a boiling temperature of the target molecule at 1 atm pressure. In some embodiments, conditions that promote crystal formation of the target molecule include a temperature of an environment external to the solution and the matrix, of between or equal to 20° C. and 30° C. and a pressure of the external environment of 1 atm. In some embodiments, conditions that promote crystal formation of the target molecule include a temperature of an environment external to the solution and the matrix, of about 25° C. and a pressure of the external environment of 1 atm. In some embodiments, the temperature of the surrounding environment external to the solution and the matrix is room temperature. Other ranges are also possible.

In some embodiments, a method comprises preparing a solution of a target molecule (e.g., step 102 of FIG. 1, step 202 of FIG. 2). In some embodiments, preparing a solution comprises exposing a target molecule to a solvent or a solvent mixture of two or more solvents under conditions that promote dissolution of the target molecule. In some embodiments, preparing a solution comprises mixing a target molecule with a solvent or a solvent mixture of 2 or more solvents. In some embodiments, conditions that promote dissolution of a target molecule include a temperature of between or equal to a melting temperature and a boiling temperature of the target molecule at 1 atm pressure. In some embodiments, conditions that promote dissolution of a target molecule include a temperature of between or equal to a melting temperature and a boiling temperature of the solvent or solvent mixture at 1 atm pressure. Other ranges are also possible.

In some embodiments, a method comprises functionalizing a surface of a matrix (e.g., step 106 of FIG. 1, step 206 of FIG. 2). In some embodiments, a method comprises functionalizing a surface of a matrix with one or more functional groups that facilitate crystal formation of a target molecule from a solution comprising the target molecule. In some embodiments, a method comprises functionalizing a surface of a matrix with one or more functional groups that act as an antisolvent for a target molecule. Methods of functionalization of a matrix with a functional group will be known to those of ordinary skill in the art and are specific to chemical compositions of a matrix and a chemical comprising the functional group, which chemical is used to attach the functional group to the matrix. Illustrative chemical compositions are disclosed herein. In some embodiments, a method comprises functionalizing a surface of a silica matrix with a commercially available alkoxysilane or halosilane.

In some embodiments, a method comprises eluting at least a portion of one or more crystals from a matrix (e.g., step 110 of FIG. 1, step 210 of FIG. 2). In some embodiments, eluting comprises re-dissolving at least a portion of one or more crystals in a solvent or solvent mixture of 2 or more solvents, wherein the target molecule has a solubility of 10, 20, 30, 40, 50, 60, 70, 80, 90, 100, 200, 300, 400, 500, 600, 700, 800, 900, or 1,000 micrograms/mL, e.g., 10-1000 micrograms/mL in the solvent or solvent mixture at 25° C. In some embodiments, eluting comprises re-dissolving at least a portion of one or more crystals in a solvent or solvent mixture of 2 or more solvents, wherein the target molecule has a solubility of between or equal to 50 micrograms/mL and 500 micrograms/mL in the solvent or solvent mixture at 25° C. In some embodiments, eluting comprises re-dissolving at least a portion of one or more crystals in a solvent or solvent mixture of 2 or more solvents, wherein the target molecule has a solubility of between or equal to 100 micrograms/mL and 300 micrograms/mL in the solvent or solvent mixture at 25° C. In some embodiments, eluting comprises flowing a solvent or solvent mixture through at least a portion of a matrix. Other ranges are also possible.

In some embodiments, a method comprises harvesting a matrix from a container, e.g., after crystallization and elution, or after crystallization and before elution. In some embodiments, a method comprises washing a matrix, e.g., after crystallization and elution, or after crystallization and before elution. In some embodiments, a method comprises drying a matrix (e.g., by vacuum or pressurized gas (e.g., nitrogen, air)), e.g., after crystallization and elution, or after crystallization and before elution.

In some embodiments, systems are provided. In some embodiments, systems for forming crystals (e.g., crystals comprising a target molecule) are provided. In some embodiments, the systems comprise a container comprising a matrix and a solution of the target molecule. In some embodiments, a system comprises a matrix described herein. For example, in some embodiments, a system comprises a functionalized nanoporous matrix (e.g., an antisolvent-functionalized nanoporous matrix).

### Solution

In some embodiments, methods and systems described herein comprise a solution of a target molecule. In some embodiments, a solution is at an undersaturated state with respect to a target molecule. In some embodiments, an undersaturated state of a solution with respect to a target molecule involves the target molecule at between or equal to 40% and 99% of an equilibrium concentration in a solution. In some embodiments, an undersaturated state of a solution with respect to a target molecule involves the target molecule at between or equal to 50% and 99% of an equilibrium concentration in the solution. In some embodiments, an undersaturated state of a solution with respect to a target molecule involves the target molecule at between or equal to 60% and 99% of an equilibrium concentration in the solution. In some embodiments, an undersaturated state of a solution with respect to a target molecule involves the target molecule at between or equal to 80% and 90% of an equilibrium concentration in the solution. In some embodiments, an undersaturated state of a solution with respect to a target molecule involves the target molecule at about 55%, 60%, 65%, 70%, 75%, 80%, 85%, or 90% of an equilibrium concentration in the solution. Other ranges are also possible.

In some embodiments, a solution comprises a particular target molecule. In some embodiments, a target molecule is an active pharmaceutical ingredient (API), or an intermediate in a synthesis of an API, or an intermediate in a chemical synthesis in which a solvent switch is involved. In certain embodiments, a target molecule is an active pharmaceutical ingredient (API). In some embodiments, a target molecule is a protein. Non-limiting examples of proteins include monoclonal antibodies, small protein therapeutics, enzymes, enzyme mimetics, clotting factors, growth factors, interferons, interleukins, and thrombolytics.

In some embodiments, a solution comprises a solvent or a solvent mixture of 2 (two) or more solvents. In some embodiments, a solution comprises a solvent or a solvent mixture of 2 or more solvents, wherein a target molecule has a solubility of between or equal to 50 micrograms/mL and 500 micrograms/mL in the solvent or solvent mixture at 25° C. In some embodiments in which a target molecule is fenofibrate (FEN), a solvent or solvent mixture comprises ethyl acetate. In some embodiments in which a target molecule is diphenhydramine hydrochloride (DPH), aspirin (ASA), or nicotinamide (NIC), a solvent or solvent mixture comprises isopropyl alcohol. In some embodiments in which a target molecule is acetaminophen (APAP), a solvent or solvent mixture comprises water.

In some embodiments, a solution comprises an impurity, having a solubility of between or equal to 50 micrograms/mL and 500 micrograms/mL in the solvent or solvent mixture at 25° C. Other ranges are also possible.

### Nanoporous Matrix

In some embodiments, a method involves exposing a solution to a matrix. In some embodiments, a system comprises a matrix.

In some embodiments, a matrix has a high specific surface area. In some embodiments, a matrix has a specific surface area of between or equal to 100 m²/gram and 500 m²/gram, e.g., about 100 m²/gram, about 200 m²/gram, about 300 m²/gram, about 400 m²/gram, or about 500 m²/gram, or between or equal to 100 and 200 m²/gram, or between or equal to 200 and 300 m²/gram, or between or equal to 300 and 400 m²/gram, or between or equal to 400 and 500 m²/gram. In some embodiments, a matrix has a specific surface area of between or equal to 160 m²/gram and 180 m²/gram.

In some embodiments, a matrix is porous. In some embodiments, a matrix is nanoporous. In some embodiments, a matrix has an average pore size of at most 100 nm, at most 90 nm, at most 80 nm, at most 70 nm, at most 60 nm, at most 50 nm, at most 40 nm, at most 30 nm, at most 29 nm, at most 28 nm, at most 27 nm, at most 26 nm, or at most 25 nm. In some embodiments, a matrix has an average pore size of at least 1 nm, at least 2 nm, at least 3 nm, at least 4 nm, at least 5 nm, at least 10 nm, at least 15 nm, at least 20 nm, at least 21 nm, at least 22 nm, at least 23 nm, or at least 24 nm. Combinations of the above-referenced ranges are also possible (e.g., between or equal to 1 nm and 100 nm, between or equal to 1 nm and 50 nm, between or equal to 1 nm and 25 nm, between or equal to 10 nm and 100 nm, or between or equal to 1 nm and 10 nm). In some embodiments, a matrix has an average pore size of between or equal to 1 nm and 100 nm. In some embodiments in which a target molecule is a protein, a matrix has an average pore size of between or equal to 10 nm and 100 nm. In some embodiments, a matrix has an average pore size of between or equal to 1 nm and 50 nm. In some embodiments, a matrix has an average pore size of between or equal to 1 nm and 25 nm. In some embodiments, a matrix has an average pore size of between or equal to 5 nm and 20 nm. In some embodiments, a matrix has an average pore size of 7 nm. Other ranges are also possible.

In some embodiments, a matrix comprises one or more materials. In some embodiments, a matrix comprises one or more materials capable of being functionalized. In some embodiments, a matrix comprises silica, alumina, anodic aluminum oxide (AAO), a zeolite, or carbon, or a combination thereof.

In some methods and systems described herein, a matrix is functionalized on at least one surface of the matrix. In some methods and systems described herein, a matrix is functionalized within at least some pores in the matrix. In some methods and systems described herein, a matrix is functionalized with an antisolvent group, as is described further herein. In some embodiments, a matrix is functionalized with a plurality of functional groups in the form of a self-assembled monolayer (SAM). Without wishing to be bound by theory, in some embodiments, a functional group or a self-assembled monolayer of functional groups may lower an energy barrier to nucleation of crystals of a target molecule. In some embodiments, a matrix is functionalized with a plurality of different antisolvent groups.

In some embodiments, a surface of a matrix is functionalized with one or more functional groups having a particular surface area percent coverage. In some embodiments, a surface area percent coverage of one or more functional groups on a surface of the matrix is between or equal to 25% and 100%. In some embodiments, a surface area percent coverage of one or more functional groups on a surface of the matrix is between or equal to 25% and 50%, e.g., about 25%, about 30%, about 35%, about 40%, about 45%, or about 50%. In some embodiments, a surface area percent coverage of one or more functional groups on a surface of the matrix is between or equal to 50% and 100%, e.g., about 55%, about 60%, about 65%, about 70%, about 75%, about 80%, about 85%, about 90%, or about 95%.

In some embodiments, a surface of a matrix is functionalized with an alkyl group. In some embodiments, a surface of a matrix is functionalized with an unbranched alkyl group. In some embodiments, a surface of a matrix is functionalized with an unbranched alkyl group between or equal to 3 carbons and 12 carbons in length. In some embodiments, a surface of a matrix is functionalized with an octyl group.

In some embodiments, a surface of a matrix is functionalized with an aryl group (e.g., phenyl group). As a non-limiting example, a surface of a matrix (e.g., silica) may be functionalized with a 2-Diphenylphosphinoethyl group.

In some embodiments, a surface of a matrix is functionalized with a carboxyl (—COOH) group. As a non-limiting example, a surface of a matrix (e.g., silica) may be functionalized with propylcarboxylic acid, 1-aminopropyl(3-oxobutanoic acid), or a 3-carboxypropyl group.

In some embodiments, a surface of a matrix is functionalized with a cyano (—CN) group. As a non-limiting example, a surface of a matrix (e.g., silica) may be functionalized with a 2-cyano group.

In some embodiments, a surface of a matrix is functionalized with a hydroxyl (—OH) group. As a non-limiting example, a surface of a matrix (e.g., silica) may be functionalized with 3-propylsufonic acid.

In some embodiments, a surface of a matrix is functionalized with an amine group. As a non-limiting example, a surface of a matrix (e.g., silica) may be functionalized with propylamine.

In some embodiments, a surface of a matrix is functionalized with an alkenyl (e.g., vinyl) group. As a non-limiting example, a surface of a matrix (e.g., silica) may be functionalized with triethoxyvinylsilane.

In some embodiments, a surface of a matrix is functionalized with a thiol (—SH) group. As a non-limiting example, a surface of a matrix (e.g., silica) may be functionalized with propylthiol.

In some embodiments, a surface of a matrix is functionalized with a polyethylene glycol (PEG) group.

In some embodiments, a matrix (e.g., an antisolvent-functionalized nanoporous matrix) is configured and arranged to promote crystallization of a target molecule from a solution. In some embodiments, a matrix is configured as a packed bed in a portion of a container. In some embodiments, a matrix is configured as a monolithic body in a portion of the container.

In some embodiments, a matrix comprises one or more particles. In some embodiments, one or more matrix particles have a particular average diameter. In some embodiments, one or more matrix particles have an average diameter of between or equal to 100 nm and 200 microns. In some embodiments, matrix particles have an average diameter of between or equal to 1 micron and 50 microns, e.g., between or equal to 1 micron and 10 microns. In some embodiments, matrix particles have an average diameter of 7 microns. Other ranges are also possible.

In some embodiments, systems described herein comprise a container. In some embodiments, methods described herein are carried out in a container. In some embodiments, a container is cylindrical. In some embodiments, a container comprises stainless steel. In some embodiments, a container comprises a matrix described herein. In some embodiments, a system comprises a container comprising a solution of a target molecule described herein. In some embodiments, a system comprises a first container comprising a matrix described herein and a second container comprising a solution of a target molecule described herein.

### Definitions

As used herein, a “crystal” is given its ordinary meaning in the art, i.e., a solid comprising atoms forming a periodic arrangement along one or more dimensions (e.g., along one dimension, along two dimensions, along 3 dimensions).

As used herein, a “solution” is given its ordinary meaning in the art, i.e., a composition comprising one or more solvents and one or more dissolve solutes (e.g., target molecule). Some solutions herein are fluids.

As used herein, a “fluid” is given its ordinary meaning, i.e., a liquid or a gas. A fluid cannot maintain a defined shape and will flow during an observable time frame to fill the container in which it is put. Thus, the fluid may have any suitable viscosity that permits flow. If two or more fluids are present, each fluid may be independently selected among essentially any fluids (liquids, gases, and the like) by those of ordinary skill in the art.

As used herein, a solution is at an “undersaturated” state “with respect to” a component provided that a concentration of the component in solution is less than an equilibrium concentration of solute in solution at the conditions of temperature, pressure, and/or compositions etc. to which the solution is exposed. In some embodiments, a solution is at an undersaturated state with respect to a component provided that it is possible to dissolve more of the component in the solution at the conditions and/or compositions of temperature, pressure, functional groups, etc. to which the solution is exposed.

As used herein, a solution is at a “saturated” state “with respect to” a component provided that a concentration of the component in solution is equal to an equilibrium concentration of solute in solution at the conditions of temperature, pressure, and/or compositions etc. to which the solution is exposed. In some embodiments, a solution is at a saturated state with respect to a component provided that it is not possible to dissolve more of the component in the solution at the conditions and/or compositions of temperature, pressure, functional groups, etc. to which the solution is exposed.

As used herein, a solution is at a “supersaturated” state “with respect to” a component provided that a concentration of the component in solution is greater than an equilibrium concentration of solute in solution at the conditions of temperature, pressure, and/or compositions etc. to which the solution is exposed. In some embodiments, a solution is at a supersaturated state with respect to a component provided that it is not possible to dissolve more of the component in the solution at the conditions and/or compositions of temperature, pressure, functional groups, etc. to which the solution is exposed and at least some of the component contacts the solution and remains undissolved in solution, e.g., one or more undissolved crystals of the component contact the solution.

A solution in a state before interaction with a matrix is also referred to herein as a “parent solution”.

As used herein, a “nanoporous matrix” refers to a matrix having an average pore size (e.g., diameter, width) of less than about 1 micron. In embodiments where pores of a porous matrix (e.g., a nanoporous matrix) are spherical, average pore size may be determined by a number average diameter of the pores. In embodiments where pores of a porous matrix are oblong or of an irregular shape, average pore size may be determined by a number average diameter of a plurality of pores, wherein each pore is first calculated to have an equivalent sphere with a diameter equal to an average diameter of the oblong shaped pore. An average pore size of a porous matrix may be measured according to any method known in the art. For example, an average pore size may be determined by image analysis, e.g., of scanning electron microscopy images.

As used herein, a “functionalized” entity (e.g., a functionalized matrix, a functionalized nanoporous matrix) refers to the presence of one or more chemical functional groups on a surface of the entity. The one or more functional groups may be attached covalently, by a gold-thiol attachment, by metallic bonding, by ionic bonding, by hydrophobic interactions, by dipole-dipole interactions, or by another inter-molecular interaction. An entity may be functionalized in a manner such that the functional group is not removed from the surface of the entity during combination of the functionalized entity with a solution of a target molecule. In some embodiments, one or more functional groups may be attached to a surface of a matrix in an irreversible manner (e.g., by a covalent bond).

As used herein, a “monolayer” refers to a layer that is one molecule in thickness (e.g., a diameter, width, thickness, length, or other appropriate dimension of a molecule depending on its orientation relative to the underlying material).

As used herein, a “self-assembled monolayer” refers to a monolayer that spontaneously forms on one or more surfaces of a matrix upon exposure (e.g., by immersion) to a liquid or gas comprising one or more molecules having one or more functional groups.

As used herein, an “antisolvent” refers to a solvent in which a target molecule has a solubility of between or equal to 1.01 times and 10,000 times less than that of a solubility of the target molecule in a solution (comprising a solvent or solvent mixture) before exposure to a matrix described herein. In some embodiments, a target molecule has at least 1.01 times less, at least 1.02 times less, at least 1.1 times less, at least 1.2 times less, or at least 1.4 times less solubility in an antisolvent than in a solution before exposure to a matrix described herein. In some embodiments, a target molecule has at most 1.6 times less, at most 1.8 times less, at most 2 times less, at most 4 times less, at most 6 times less, at most 8 times less, at most 10 times less, at most 100 times less, at most 1000 times less, or at most 10,000 times less solubility in an antisolvent than in a solution before exposure to a matrix described herein. Combinations of the above-referenced ranges are also possible (e.g., between or equal to 1.01 times less and 10,000 times less, between or equal to 1.01 times less and 1000 times less, between or equal to 1.01 times less and 2 times less). Other ranges are also possible.

As used herein, an “antisolvent group” refers to a group that functions as an antisolvent for a target molecule. In some embodiments, an antisolvent group reduces a solubility of a target molecule, within a pore of a matrix functionalized with the antisolvent group, by between or equal to a 0.1% and 99.99% reduction in solubility relative to its solubility in a solution before exposure to the matrix. In some embodiments, a target molecule has at least a 0.1%, at least a 0.2%, at least a 1%, at least a 2%, or at least a 4%, reduced solubility within a pore of a matrix (a surface of which pore is functionalized with an antisolvent group) relative to its solubility in a solution before exposure to the matrix. In some embodiments, a target molecule has at most a 99.99%, at most a 99.9%, at most a 99%, at most a 98%, at most a 90%, at most an 80%, at most a 70%, at most a 60%, at most a 50%, at most a 40%, at most a 30%, at most a 20%, at most a 10%, at most an 8%, or at most a 6% reduced solubility within a pore of a matrix (a surface of which pore is functionalized with an antisolvent group) relative to its solubility in a solution before exposure to the matrix. Combinations of the above-referenced ranges are also possible (e.g., between or equal to 0.1% and 99.99% reduction, between or equal to 0.1% and 99.9% reduction, between or equal to 0.1% and 10% reduction). Other ranges are also possible.

As used herein, an “equivalent antisolvent” refers to a solvent having a functional group in common with a surface of a matrix described herein. As a non-limiting example, octane is an equivalent antisolvent for an octyl group functionalizing a surface of a matrix described herein.

As used herein, an “antisolvent-functionalized” entity (e.g., matrix) is functionalized with one or more antisolvent groups.

As used herein, a “packed bed” refers to a hollow container (e.g., tube, pipe) filled with a plurality of matrices having been packed together (e.g., by pressure) in at least a portion of a hollow container.

As used herein, a “monolithic body” refers to a body consisting of a single piece. By contrast, a body is not a monolithic body if it comprises one or more joints, seams, adhesives, or other means for connecting two or more pieces together to form the body. In some embodiments, a monolithic body is porous. In some embodiments, a monolithic body is nanoporous. In some embodiments, a monolithic body is rigid.

As used herein, “specific surface area” (SSA) is given its ordinary meaning in the art, i.e., the total surface area of the article per unit mass. Specific surface area may be determined by a Brunauer-Emmett-Teller (BET) analysis method, which will be known to those of ordinary skill in the art.

As used herein, in some embodiments, “average pore size” is determined as an average size of pores in a matrix without taking into account surface functionalization within pores, which in some embodiments decreases in average effective size of pores.

As used herein, a “container” or “vessel” is given its ordinary meaning, i.e., an article configured to hold one or more materials inside at least a portion of the article.

As used herein, “eluting” is given its ordinary meaning, i.e., removing one or more adsorbed components from an article by exposing the article to a solvent under conditions that promote dissolution of the one or more adsorbed components.

As used herein, “isothermal” conditions refer to a constant temperature of an environment external to a solution and/or a matrix for the duration of a process (e.g., flowing a solution through at least a portion of a matrix).

As used herein, the term “active pharmaceutical ingredient” (also referred to as a “drug”) refers to an agent that is administered to a subject to treat a disease, disorder, or other clinically recognized condition, or for prophylactic purposes, and has a clinically significant effect on the body of the subject to treat and/or prevent the disease, disorder, or condition. Active pharmaceutical ingredients include, without limitation, agents listed in the United States Pharmacopeia (USP), Goodman and Gilman's The Pharmacological Basis of Therapeutics, 10th edition, McGraw Hill, 2001; Katzung, B. (editor), Basic and Clinical Pharmacology, McGraw-Hill/Appleton & Lange, 8th edition (Sep. 21, 2000); Physician's Desk Reference (Thomson Publishing); and/or The Merck Manual of Diagnosis and Therapy, 17th edition (1999), or the 18th edition (2006) following its publication, Mark H. Beers and Robert Berkow (editors), Merck Publishing Group, or, in the case of animals, The Merck Veterinary Manual, 9th edition, Kahn, C. A. (ed.), Merck Publishing Group, 2005. Preferably, though not necessarily, the active pharmaceutical ingredient is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention. In some exemplary embodiments, an active pharmaceutical ingredient is aspirin (ASA), nicotinamide (NIC), diphenhydramine hydrochloride (DPH), acetaminophen (APAP), or fenofibrate (FEN).

A “subject” refers to any animal such as a mammal (e.g., a human). Non-limiting examples of subjects include a human, a non-human primate, a cow, a horse, a pig, a sheep, a goat, a dog, a cat or a rodent such as a mouse, a rat, a hamster, a bird, a fish, or a guinea pig.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

As used herein, the term “alkyl” is given its ordinary meaning in the art and refers to the radical of saturated aliphatic groups (“saturated” in this instance given its chemical meaning of comprising only single bonds), including straight-chain alkyl groups, branched-chain alkyl groups, cycloalkyl (alicyclic) groups, alkyl substituted cycloalkyl groups, and cycloalkyl substituted alkyl groups. In some cases, the alkyl group may be a lower alkyl group, e.g., an alkyl group having 1 to 10 carbon atoms (e.g., methyl, ethyl, propyl, butyl, pentyl, hexyl, heptyl, octyl, nonyl, or decyl). In some embodiments, a straight chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C₁ to C₁₂ for straight chain, C₃ to C₁₂ for branched chain), 6 or fewer, or 4 or fewer. Likewise, cycloalkyls may have from 3 to 10 carbon atoms in their ring structure, or 5, 6 or 7 carbons in the ring structure. Examples of alkyl groups include, but are not limited to, methyl, ethyl, propyl, isopropyl, cyclopropyl, butyl, isobutyl, t-butyl, cyclobutyl, hexyl, and cyclochexyl.

The terms “alkenyl” and “alkynyl” are given their ordinary meaning in the art and refer to unsaturated aliphatic groups (“unsaturated” in this instance given its chemical meaning of comprising a double bond or triple bond) analogous in length and possible substitution to the alkyls described above, but that contain at least one double or triple bond respectively.

The term “aryl” is given its ordinary meaning in the art and refers to aromatic carbocyclic groups, optionally substituted, having a single ring (e.g., phenyl), multiple rings (e.g., biphenyl), or multiple fused rings in which at least one is aromatic (e.g., 1,2,3,4-tetrahydronaphthyl, naphthyl, anthryl, or phenanthryl). That is, at least one ring may have a conjugated pi electron system, while other, adjoining rings can be cycloalkyls, cycloalkenyls, cycloalkynyls, aryls and/or heterocyclyls. The aryl group may be optionally substituted, as described herein. Substituents include, but are not limited to, any of the previously mentioned substituents, i.e., the substituents recited for aliphatic moieties, or for other moieties as disclosed herein, resulting in the formation of a stable compound. In some cases, an aryl group is a stable mono- or polycyclic unsaturated moiety (“unsaturated” in this instance given its chemical meaning of comprising a double bond or triple bond) having preferably 3-14 carbon atoms, each of which may be substituted or unsubstituted. “Carbocyclic aryl groups” refer to aryl groups wherein the ring atoms on the aromatic ring are carbon atoms. Carbocyclic aryl groups include monocyclic carbocyclic aryl groups and polycyclic or fused compounds (e.g., two or more adjacent ring atoms are common to two adjoining rings) such as naphthyl groups.

The term “amine,” as used herein, refers to a primary (—NH₂), secondary (—NHRₓ), tertiary (—NRₓR_{y}), or quaternary (—N⁺RₓR_{y}R_{z}) amine, where Rₓ, R_{y}, and R_{z} are independently an aliphatic, alicyclic, heteroaliphatic, heterocyclic, aryl, or heteroaryl moiety, as defined herein. Examples of amine groups include, but are not limited to, methylamine, dimethylamine, ethylamine, diethylamine, methylethylamine, iso-propylamine, piperidine, trimethylamine, and propylamine.

As used herein, the term “hydroxyl” or “hydroxy” refers to the group —OH.

As used herein, the term “thiol” or “thio” refers to the group —SH.

The following examples are intended to illustrate certain embodiments of the present disclosure, but do not exemplify the full scope of the disclosure.

### EXAMPLES

Some embodiments of this disclosure involve systems and methods of forming crystals from undersaturated solution using functionalized nanoporous matrices.

Some embodiments of this disclosure pertain to forming crystals of organic compounds (an example of a target molecule), focusing on active pharmaceutical ingredients (APIs), from solutions at an undersaturated state with respect to the target molecule using a combination of confinement effects and surface functionalization effects using Zorbax®, functionalized chromatographic media (an example of a matrix described herein). Crystal formation was demonstrated in several experimental methods, one of which methods confined the media (e.g. matrix) and API solution (e.g. target molecule solution) to a sealed capillary tube chamber (e.g. container) wherein crystal formation was monitored in real time.

In some embodiments, antisolvent-functionalized nanoporous matrices are used to form crystals of an active pharmaceutical ingredient (API) that may be sensitive to changes in temperature. A solute molecule (target molecule) may be dissolved in a solvent, typically at ambient temperature. In some embodiments, antisolvent-functionalized nanoporous matrices (e.g., rather than addition of an antisolvent to the solution) generates supersaturation because the solute (target molecule) is less soluble in the presence of the antisolvent groups. In some embodiments, the choice of antisolvent group and composition of the solvent/antisolvent group mixture within a pore influences crystal size, shape, form, and yield.

In some embodiments, functionalized nanoporous matrices are configured to facilitate the formation of crystal nuclei of a target molecule from solution. In some embodiments, functionalized nanoporous matrices are configured to lower an activation energy of nucleation relative to nanoporous matrices without functional groups attached.

In some embodiments, crystal formation in confinement (e.g., in nanopores of a nanoporous matrix) resulted in production of stable nanocrystals of a controlled size. In some embodiments, crystal formation of an API was restricted to a nanoporous environment to form nanocrystals. Without wishing to be bound by theory, crystal formation of the API may have had contributions to nucleation both from confinement of a volume in which crystal formation occurs (e.g., volume of a nanopore in a matrix) and one or more functional groups on one or more surfaces of the nanopore. In some embodiments, this disclosure combines effects of crystal formation in confinement (e.g. in nanopores) with surface functionalization to produce nanocrystals of a target molecule from a solution at an undersaturated state with respect to the target molecule before exposure to nanopores and/or functionalized surfaces. Zorbax® chromatographic media (e.g. matrices) with octyl-like surface groups (e.g. functional groups) was used in some cases, to mimic a functional group interaction of alkanes, which tend to be poor solvents for organic APIs chosen for some embodiments of this disclosure. Without wishing to be bound by theory, in some embodiments, in confined volumes of nanopores in a matrix, these functional groups rendered a change in solubility of API in solution in these nanoscale volumes, providing the driving force for nucleation and crystallization.

In some embodiments, the present disclosure is directed to the dual application of crystal formation within nanopores (in confinement) and crystal formation using functionalized surfaces to facilitate crystallization of a target molecule from a solution in an undersaturated state with respect to the target molecule before interaction with the nanopores and/or functionalized surfaces. This was demonstrated, e.g., through the use of Zorbax®, functionalized chromatographic media (matrices), to form crystals of several small molecule organic compounds (target molecules). Given that parent solutions were in an undersaturated state with respect to a target molecule, when held at a fixed temperature left alone or in the presence of non-functionalized control silica media (matrices) which mimicked the surface area of the Zorbax® media, the solutions did not crystallize (without wishing to be bound by theory, because there was no driving force for nucleation). However, it was demonstrated that, in several experimental setups, the addition of the functionalized Zorbax® media induced crystallization. This media (e.g. matrix) was a porous silica having an average pore size of about 7 nm diameter, producing nanoscale solution volumes within the pores. The surface of this media was coated with an octyl-like functional group (e.g. antisolvent group), mimicking the functional groups of alkane solvents (e.g. equivalent antisolvent) which are poor solvents for the APIs chosen for this study. The confined nano scale volumes of the pores resulted in high specific surface area of the matrices, and a surface area percent coverage with functional groups was at least 25%, and thus the expected contribution of functional group interaction with a solution in this environment was high. Without wishing to be bound by theory, combined surface functionalization and confinement effects contributed to Zorbax® media acting as an antisolvent, reducing the solubility of the APIs and causing crystal formation (e.g., nucleation, growth of crystals). By contrast, in alternative methods of crystal formation in pores, a driving force was present due to antisolvent addition, evaporation, or cooling crystallization as opposed to this unique combined effect of surface functionalization and confinement. A driving force for crystallization in evaporative crystal formation is an increase in concentration of the parent solution by evaporation of the solvent. Evaporation may present issues, e.g., for temperature-sensitive target molecules. Antisolvent addition and cooling crystallization present issues described elsewhere in this disclosure.

The following experiments supported formation of nanoscale crystals of a target molecule from parent solutions (parent solutions in an undersaturated state with respect to the target molecule) in the presence of Zorbax® chromatographic media.

Submerged Loading Experiments

1) Zorbax® media was added to an undersaturated API solution and held at constant temperature with no evaporation of solvent. The media was held for several hours.

2) The media was recovered through filtration followed by a fast 10 mL cold solvent wash, dried, and analyzed.

3) The Zorbax® media was shown to have confined nanocrystals which were not present in control experiments, which control experiments were performed with un-functionalized silica media, suggesting that the Zorbax® media contributed to the formation of the nanosized crystals.

Column Loading Experiments

1) Zorbax® media (5 grams) was held in a 10 mL column and temperature of the solutions was controlled at 25° C. (e.g conditions promoting crystal formation).

2) Undersaturated API solution was flowed slowly over the column (1 mL/hour) (e.g. flowing a solution through at least a portion of a matrix) and flowthrough is collected.

3) Flowthrough analysis demonstrated that in the control experiments performed with un-functionalized media, little API was retained on the column. However when Zorbax® media was used, the concentration of API in flowthrough solution decreased dramatically.

4) The Zorbax® in the column was analyzed and nanoscale crystals were formed. No nanoscale crystals were formed when control un-functionalized media was used.

Capillary Crystallization Experiments

1) Zorbax® media was packed into 1 mm OD glass capillaries for x-ray powder diffraction (XRPD).

2) Undersaturated solutions of APIs were prepared. The capillaries were placed under slight vacuum and filled with API solution.

3) Continuous x-ray powder diffraction (XRPD) was performed on these capillaries to determine crystal formation (e.g., by evaluating crystallinity) in live time.

4) Capillaries packed with functionalized Zorbax® media showed crystallization from four different undersaturated API solutions within 1 hour, whereas capillaries packed with control media or API solution alone did not.

Prophetic Examples: Illustrative Applications of the Disclosure

Crystallization Schemes Using Zorbax® Media

Embodiments of this disclosure demonstrate a unique combination of confinement and surface functionalization to cause crystallization in undersaturated solutions. Embodiments of this disclosure may be applied to the following illustrative crystallization schemes, without limitation:

1) Crystallization of APIs: Screening of API crystallization from various solvents with this approach may be used as an alternative to antisolvent crystallization. It may elucidate differences in nucleation induction time, polymorphs, or crystal morphologies.

2) Purification of API solutions: The column setup could be particularly advantageous for the purification of API from solution containing impurities. A solution of this type could pass through a column loaded with Zorbax® media. The API would crystallize in the column and a solution of impurities could pass through the column. The API could be harvested with the Zorbax® media, re-dissolved, and crystallized again with greater purity.

3) Protein crystallization: Protein and large biologic molecule crystallization is typically challenging. There is a lack of generalized methods, and protein solutions may be dilute. This technique could be used for crystallization of undersaturated protein solutions for discovery and better understanding of crystal structure and interaction.

Matrices

In some embodiments, the small average pore size (e.g., between or equal to 1 nm and hundred nanometers) of a matrix contributed to a configuration comprising nanovolumes for crystallization. In some embodiments, functional groups on a surface within pores of the matrix altered these nanovolume environments (e.g., due to their small volumes) such that crystal formation may occur from undersaturated parent solutions. In some embodiments, average pore size and functional groups are both considered when choosing a matrix.

Non-Limiting Examples of Matrix Materials

In some embodiments, a matrix material itself (e.g., not including the one or more functional groups attached to a surface of the matrix) is inert to the one or more solvents used in crystal formation. In some embodiments, porous silica is used due to its chemically inert nature, and due to ease of production of a variety of porous silicas. These include but are not limited to controlled pore glass, Vycor®, Zorbax®. Other nanoporous materials that may be inert to solvents used in typical crystal formation methods include but are not limited to anodic aluminum, nanoporous zeolites, and nanoporous carbon. In some embodiments, a matrix material is easily functionalized; e.g., glass may be easily functionalized through a straightforward silane reaction. In some embodiments, a balance between cost of a matrix and surface area percent coverage of the matrix with functional groups is considered in choosing the matrix and functional groups.

Average Pore Size

In some embodiments, both surface area percent coverage of functional groups and average pore size of a matrix facilitate crystal formation using these methods. In some cases, Zorbax® demonstrated crystal formation of a target molecule with an average pore size of the matrix of about 7 nm. In some embodiments, an average pore size of greater than or equal to 1 nm. Typically, a nanoporous matrix has a more controlled pore volume when forming a matrix with an average pore size of greater than or equal to 1 nm, relative to an average pore size of less than 1 nm. In some embodiments, a nanoporous matrix has an average pore size of greater than or equal to 1 nm and less than or equal to 100 nm.

Functional Groups on Matrix Surfaces

In some embodiments, an octyl-like functional group was chosen for experiments because a wide range of APIs were poorly soluble in alkane solvents such as hexane, heptane, or decane. The octyl-like functional group on the Zorbax® mimicked this poor solubility, and decreased the solubility of the API in solution in the confinement volumes such that crystallization could occur from an undersaturated solution.

Prophetic Example: Functional Groups on Matrix Surfaces

Alternative functional groups, such as phenyl groups, carboxyl, or —CN groups could be used for a system in which the desired crystal product is poorly soluble in solvents with functional groups of the same. For example, a poorly water-soluble compound such as ibuprofen may demonstrate successful crystallization if a carboxyl functional group on a nanoporous surface were used to mimic the polar —OH groups present in an aqueous system. In some cases, functional groups to represent the range of solvent types typically available in pharmaceutical solubility studies can be achieved through easy silane reactions on the surfaces of glass. Non-limiting available surface groups that could be made to project from the surface of porous glass matrices through organosilane chemistry include amine, carboxyl, vinyl, or thiol groups effectively spanning a variety of hydrophobic and hydrophilic functional group types.

Materials and Methods:

Porous Matrices

Zorbax® functionalized chromatographic media was obtained from Agilent in bulk packing form. This was porous silica functionalized with an octyl-like group (n-octyldimethylsilane). The average listed grain size of the beads was 7 microns and the nominal average pore size was 7 nm in diameter with a nominal specific surface area of between or equal to 160 m²/gram and 180 m²/gram. As a control, un-functionalized controlled pore glass (CPG) was purchased from Prime Synthesis (Aston, Pa., USA). This CPG was a fumed silica with controlled average pore size of approximately 12 nm. The grain size of this CPG material was about 100 microns.

Acetaminophen (APAP) was purchased from Sigma-Aldrich (BioXtra>99.0%, Lot #SLBR2060V). Aspirin (ASA) and nicotinamide (NIC) were purchased from Sigma Life Science (USP grade, Lot #MKBQ8444V and >99.5%, Lot #BCBF9698V, respectively). Diphenhydramine hydrochloride (DPH) was purchased from BeanTown Chemical (>99.0%). Fenofibrate (FEN) was obtained from Xian Shunyi Bio-chemical Technology Company (Shaanxi, China). Solvents were purchased at ACS grade or higher purity from Fisher Scientific (Waltham, Mass., USA).

DSC and TGA Analysis

Thermogravimetric analysis (TGA) was performed on a Q500 instrument from TA instruments (Newcastle, Del., USA) connected with a nitrogen gas cylinder to maintain a flow rate of 25 mL/min to maintain an inert gas environment in the sample chamber. Between or equal to 5 mg and 10 mg of sample were loaded on platinum sample pans from TA Instruments. The samples were allowed to equilibrate at 30° C. and then heated at 10° C./min to 300° C.

A Q2000 instrument from TA instruments was used for the differential scanning calorimetry (DSC) analysis. Inert atmosphere environment was maintained in the sample chamber using a nitrogen gas cylinder set to a flow rate of 50 mL/min. An extra refrigerated cooling system (RCS 40, TA Instruments) was used to widen the available temperature range to between or equal to −40° C. and 400° C. Tzero® pans and lids were used with ˜5 mg of sample. A heating rate of 10° C./min was applied and the samples were scanned from −20° C. to 200° C.

XRPD Analysis

XRPD was performed in a capillary setup using a PANalytical X'Pert PRO (Almelo, the Netherlands) diffractometer at 45 kV with an anode current of 40 mA. The instrument had a PW3050/60 standard resolution goniometer and a PW3373/10 Cu LFF DK241245 X-ray tube.

Samples were loaded in capillary tubes (further description herein) and aligned on a goniometer capillary holder in stage capillary spinner mode with a focused point incident beam using a Cu Si focusing mirror. Settings on the incident beam path included: soller slit 0.04 rad, mask fixed 10 mm, programmable divergence slit and fixed ½° anti-scatter slit. Settings on the diffracted beam path include: soller slit 0.04 rad and ⅛° anti-scatter slit. The scan was set as a continuous scan: 2θ angle between 2° and 90°, step size 0.0167113° and a time per step of 15.240 seconds.

Submerged Loading Experiment

In initial experiments, an undersaturated solution of fenofibrate in ethyl acetate was made at 25° C. Fenofibrate was highly soluble in ethyl acetate with a saturation solubility of about 650 mg/mL at 25° C., as determined by gravimetric analysis of the filtrate of a stirred slurry. The undersaturated solution was prepared at 550 mg/mL. One gram of Zorbax® was added to a 20 mL scintillation vial, and 10 mL of the fenofibrate solution was pipetted on top. The vial was sealed and held for 6 hours at 25° C. A second vial was made in the same way using the control CPG instead of the Zorbax® material. After the 6 hours, the vials were poured over a paper filter with vacuum and immediately filtered and rinsed with 5 mL cold ethanol to remove any residual API solution. The resulting powder was dried and analyzed with DSC and TGA. The experiments were repeated for initial fenofibrate concentrations in ethyl acetate of 450, 350, and 250 mg/mL. All experiments were repeated in triplicate.

Column Loading Experiment

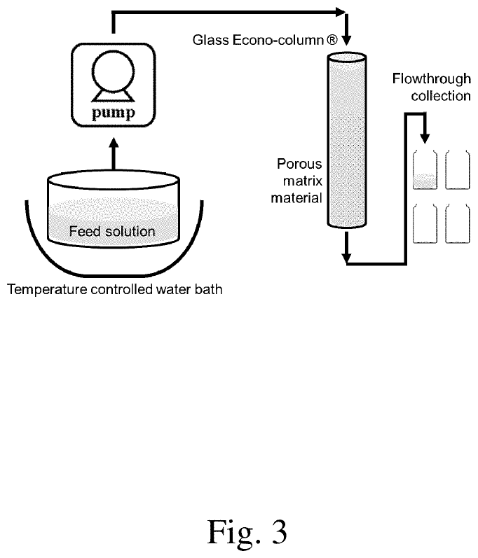

A low-pressure glass column was purchased from Bio-Rad Laboratories (Econo-Column®) that was 1.0 cm in diameter and 10 cm in length with a fill volume of about 8 mL. The column contained polypropylene end fittings and a porous polymer bed support at the bottom to retain fine particles. A feed solution bottle was placed in a water bath maintained at 25° C. (Thermo Scientific NESLAB RTE, Waltham, Mass., USA). Peristaltic pumps (Masterflex P/S, Thermo Scientific) with Viton tubing (Cole-Parmer, Vernon Hills, Ill., USA) were used for solution transfer. The column was compatible with isopropyl alcohol at room temperature. A feed solution of was prepared of diphenhydramine hydrochloride in isopropyl alcohol 25° C. at 30 mg/mL. The column was packed with 5 grams of Zorbax@, or CPG for the control case. This solution was pumped to the column at 1.0 mL/min. The column was run for 60 minutes and the flowthrough solution was collected. Two mL of solution was collected at timepoints 0, 20, and 40 minutes, and the total collected volume was sampled at the end of the run at 60 minutes. Gravimetric analysis was used to determine the concentration of DPH in solution. At the end of the run, the column was flushed with 10 mL of isopropyl alcohol at 0° C. followed by air. Samples of the dried column packing were collected and analyzed with TGA and DSC. Two runs each of both the Zorbax® and the CPG packing were performed. A schematic of an apparatus used in some experiments described herein is shown in FIG. 3.

Capillary Crystal Formation Experiment

Slightly undersaturated solutions of several APIs were prepared at 25° C. Saturation solubilities of these compounds in the chosen solvents were first found by stirring a slurry of the API in solvent at 25° C. for several days, filtering the solution, and performing gravimetric analysis. These experiments were repeated in triplicate. A saturation solubility of acetaminophen in water was found to be 15.5 mg/mL. An undersaturated solution of 12.0 mg/mL acetaminophen in water was prepared. A saturation solubility of aspirin in isopropyl alcohol was determined to be 90.0 mg/mL. An undersaturated solution of 75.0 mg/mL aspirin in isopropyl alcohol was prepared. A saturation solubility of diphenhydramine hydrochloride in isopropyl alcohol was found to be 38.0 mg/mL and an undersaturated solution of 30.0 mg/mL was prepared. Finally, a saturation solubility of nicotinamide in isopropyl alcohol was found to be 46.5 mg/mL and a solution of 38.0 mg/mL was prepared.

Glass capillary tubes were purchased from Hampton Research (Aliso Viejo, Calif.). The tubes were made of glass #50, with a 1.0 mm OD, wall thickness of 0.01 mm and length of 80 mm. The capillary tubes were packed with Zorbax® or CPG material by scooping powder into the tip, placing the capillary in a larger diameter glass pipette, and tapping to allow gravity to act on the powder to settle. Once packed, the tubes were suctioned at the open end with a 1-mL plastic syringe, using a small piece of rubber tubing to adapt the fit. The filled capillary was then immediately dipped into the undersaturated API solutions to allow the solution to fill the tube. Molten was used to plug the end of the capillary, and the tip was then heated in a Bunsen burner flame to seal. Additional capillaries were filled with liquid API solution alone.

Results:

Submerged Loading Experiment

In some experiments, an undersaturated API solution was used and no solvent was allowed to evaporate, and thus maintained the same undersaturated state under which no crystal nucleation occurred in the parent solution. Therefore, without wishing to be bound by theory, in some embodiments, any driving force for crystallization originated from interaction with functionalized porous silica matrices. Additionally, some driving force for crystallization could be expected in the handling of the material at the end of the experiment. Wash and dry steps were used to minimize any crystallization from evaporation of remaining solution.

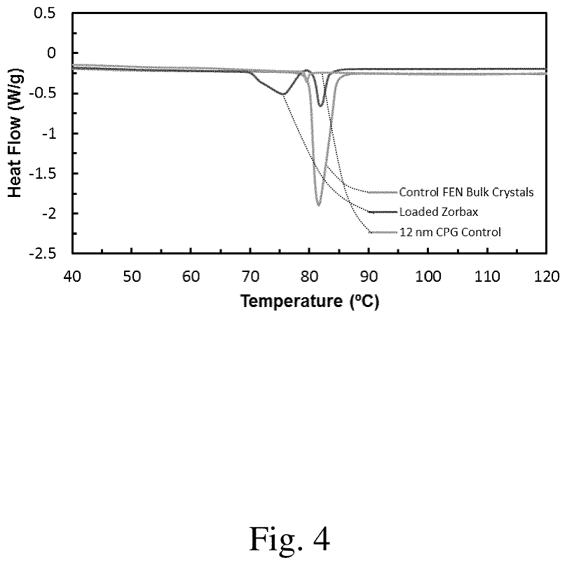

Samples loaded onto the Zorbax® material (e.g., FIG. 4) had two distinct melting peaks, one at a depressed melting point indicative of smaller sized crystals confined to the pores of the material, and one at the bulk melting temperature. A DSC scan of a control material (e.g., FIG. 4) showed a very small peak at the bulk FEN melting point, indicating that the crystals formed were of micron size and likely formed on the surface of the porous glass material as a result of the wash and dry steps.

FIG. 4 shows DSC scans of porous glass material (matrix) loaded with a submerged basket from an undersaturated solution of FEN (e.g. target molecule) in ethyl acetate (550 mg/mL; e.g. solvent). The Zorbax® material showed two distinct melting points indicating the presence of crystals in the pores.

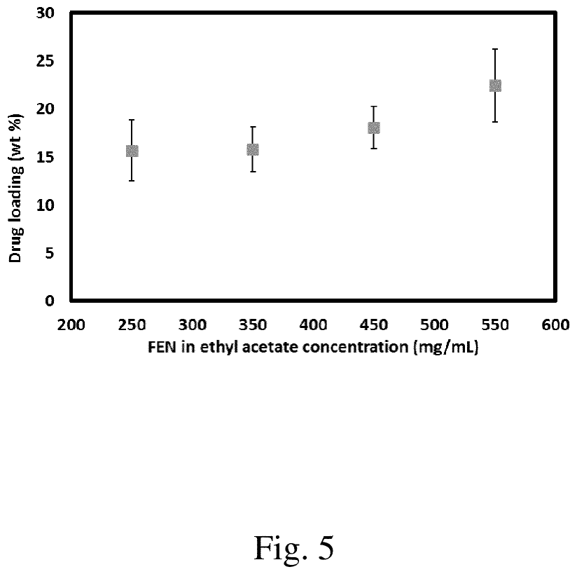

When Zorbax® was used as opposed to un-functionalized CPG, the average loading as determined by TGA was significantly higher at an average of 22.4 wt %±3.8 wt % for the samples loaded from solutions of 550 mg/mL FEN in ethyl acetate. The concentration of FEN in the loading solution was varied over several more undersaturated values, shown in FIG. 5. The resultant loading of fenofibrate on the collected powders decreased with decreasing initial API concentration. Without wishing to be bound by theory, this may indicate that initial API concentration is correlated with the driving force for crystal formation. In combination with the two melting peaks seen in the DSC, this indicated that it was likely crystal formation was occurring as a result of the presence of the functionalized nanoporous Zorbax® material as opposed to just during the wash and dry steps of the process.

Column Loading Experiment

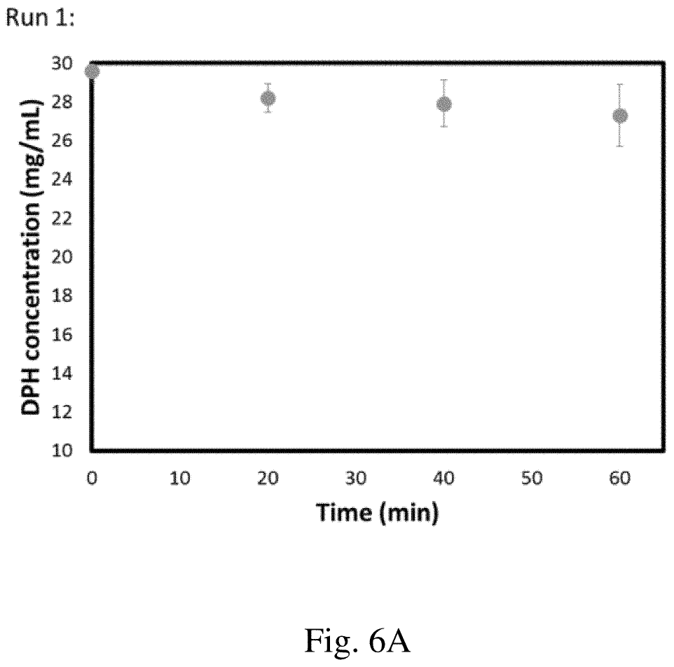

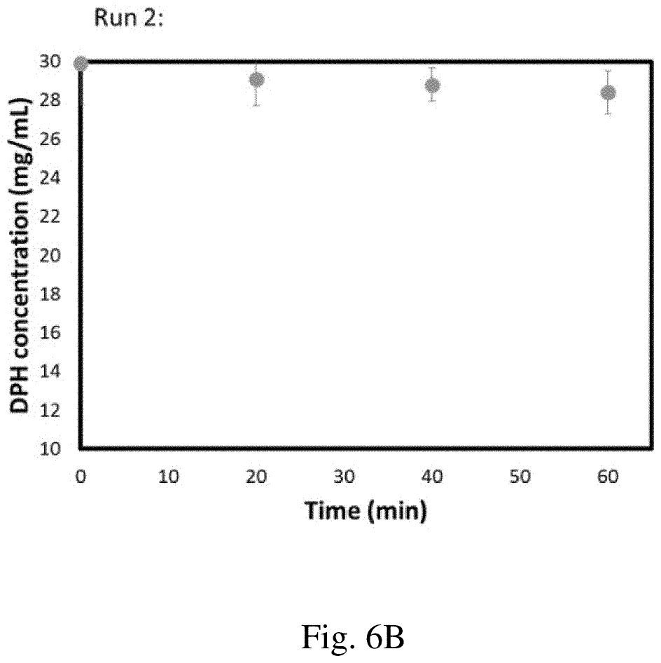

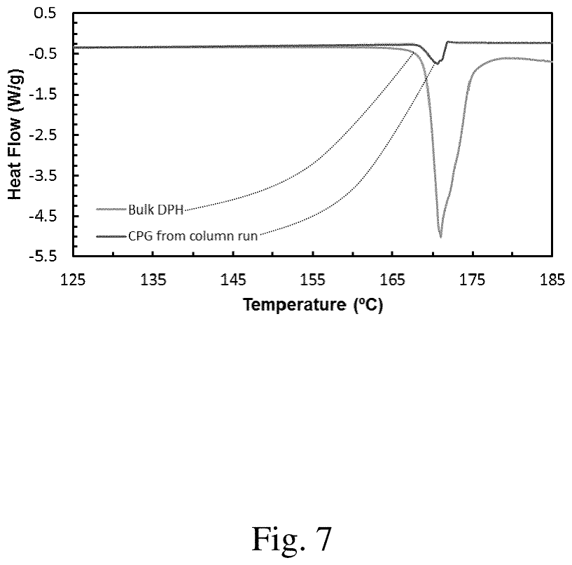

Flowthrough collected from two runs of the column setup performed using un-functionalized controlled pore glass showed little change in the concentration of API in solution over the duration of an experiment. A solution fed to a column was undersaturated DPH in isopropyl alcohol, so it was therefore not surprising that in the well-controlled environment of the column, no evaporation or temperature changes occurred to provide the driving force for nucleation of crystals. FIG. 6A and FIG. 6B show the flowthrough concentrations in each run of the CPG material. Concentrations of DPH in isopropyl alcohol varied little with time from the original 30 mg/mL value, average 28.2 mg/mL and 29.1 mg/mL at the end of the two runs. The powder (e.g. matrix) was also extracted at the end of the column run. This material showed less than 2 wt % DPH present by TGA. The DSC scan showed only the slightest presence of bulk form DPH, likely crystallized on the surface of the porous glass or column during the wash and dry steps.

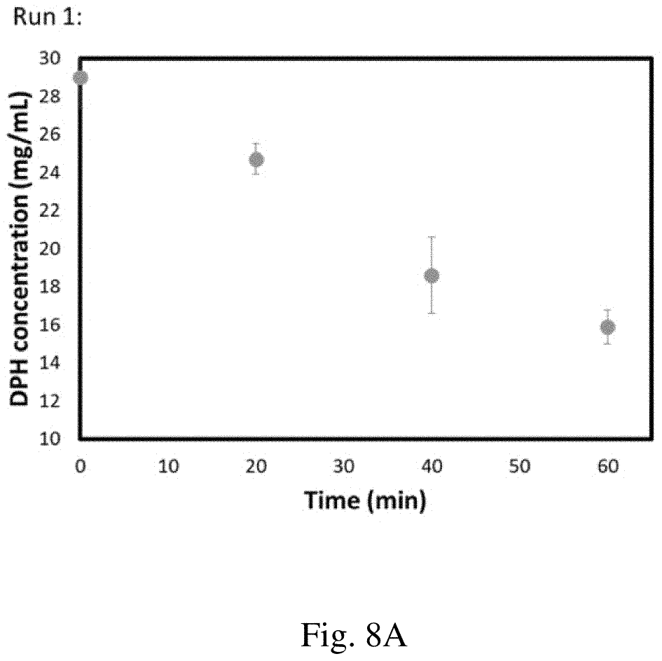

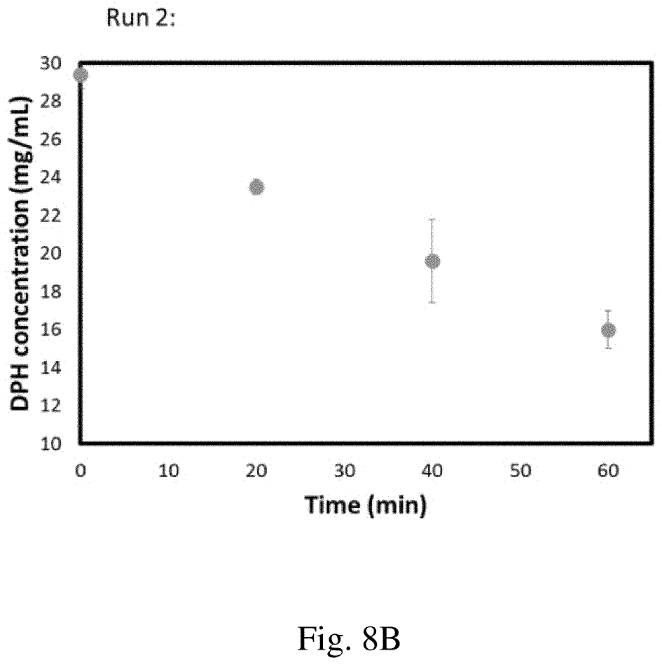

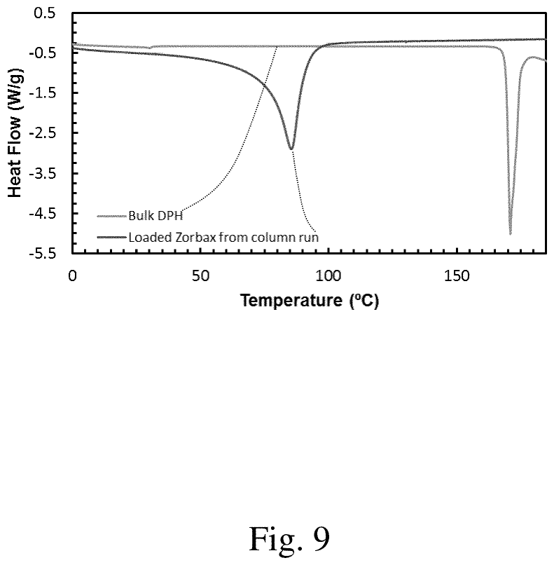

When column runs were performed with Zorbax® chromatographic media, flowthrough concentration of DPH in solution changed dramatically over the course of an experiment. FIG. 8A and FIG. 8B show a decrease in concentration in the flowthrough, from an initial concentration of 30.0 mg/mL to an average end concentration of 15.9 mg/mL and 16.0 mg/mL in the two runs, as measured in the collected pool of flowthrough at the end of the run. This significant decrease in concentration indicated DPH retention on the column (e.g., in the matrix). The powder (e.g. matrix) was extracted from the column and analyzed with thermogravimetric analysis (TGA) and DSC. DSC (FIG. 9) showed a single large peak at a decreased melting temperature, indicating that the majority of DPH crystals formed in the column were confined to the pores of the matrix. TGA indicated loadings of 14.7 wt % and 15.4 wt % in the two runs. A significant amount of the DPH in solution was crystallized in the column due to the presence of the functionalized porous silica media as opposed to the control un-functionalized silica. A mass balance was performed on each of these runs. Of the original 30 mg/mL solution flowed at 1 mL/minute for 60 minutes, the total original API present was calculated (1800 mg). The average TGA determined loading for the runs corresponded to a yield of 41% and 43% respectively. Flowthrough was analyzed to account for the remainder of the material, and it was found that for Run 1 the mass balance summing the loaded and flowthrough API accounted for 94% of the total material and in Run 2, 96% was accounted for. Considering that the sampling of the loaded Zorbax® was an average over the entire column and some API material may have been trapped in the inlet and outlet apertures, these mass balances were successfully closed. While the <50% yields occurred with one run of solution through a column, the flowthrough material may be easily recycled and passed through another column or the same column in a recycle loop, thereby increasing the expected yield. Furthermore, one application for this method, as indicated elsewhere herein, may be in the purification of APIs. If an API that may interact with selected functional groups preferentially forms crystals on a column (e.g., in a matrix), flowthrough solution may be enriched in impurity, and purified API may be collected in the column. The column may then be eluted with fresh solvent to achieve a high purity API solution dissolved off the column.

Capillary Crystallization Experiment 1

Results of some example experiments described herein indicated that the presence of the functionalized Zorbax® material contributed to crystal formation whereas un-functionalized CPG did not. As another approach to address the question of whether the crystal formation seen in the Zorbax® samples was attributable to the Zorbax® itself, a system was isolated from other possible causes of driving force for crystallization (e.g., cooling, evaporation, or antisolvent (vs. antisolvent groups)) and monitor crystal formation in a non-invasive way. Zorbax® material was isolated to a glass capillary tube and loaded with API solution, after which the tube was sealed. Crystal formation was then monitored with x-ray powder diffraction (XRPD). When the same procedure was carried out with un-functionalized CPG, no changes were seen in the XRPD over time, with just an amorphous background signal from the silica. These results were obtained for four API solutions. Furthermore, capillaries that were filled with API solution only also did not crystallize.

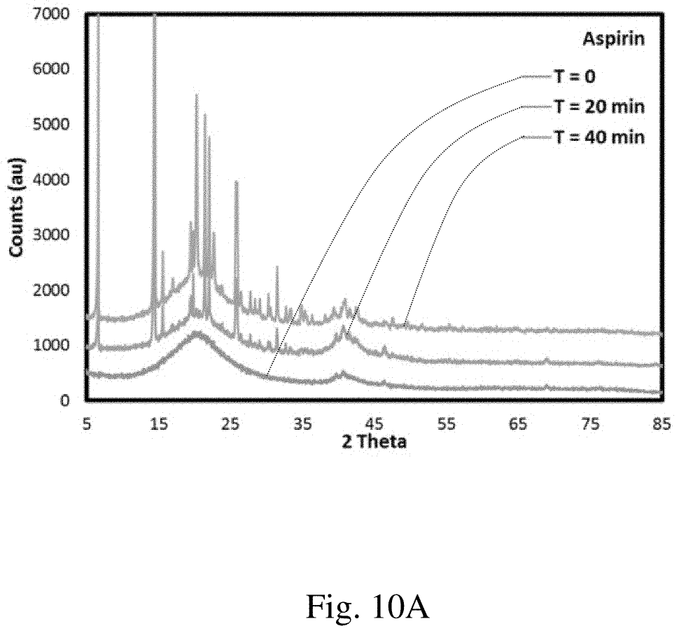

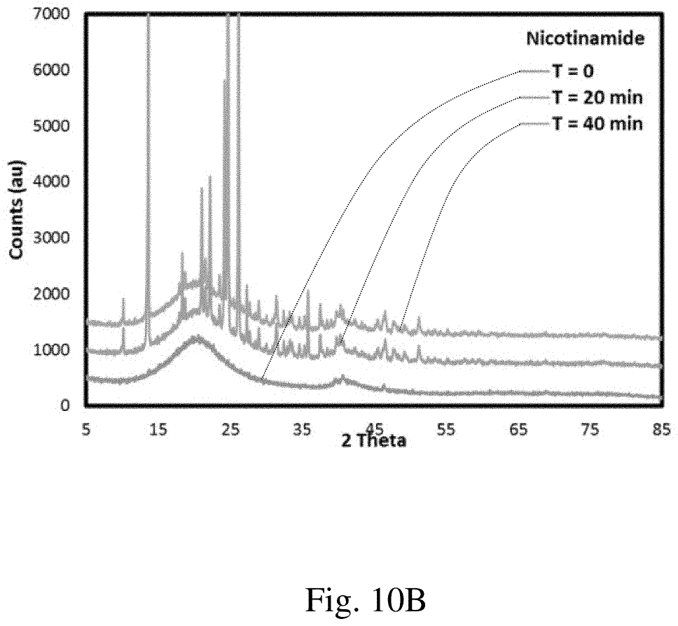

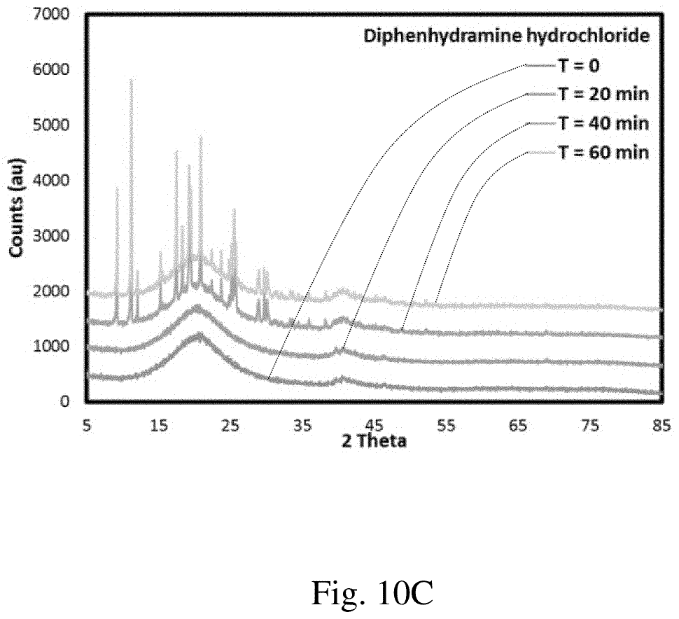

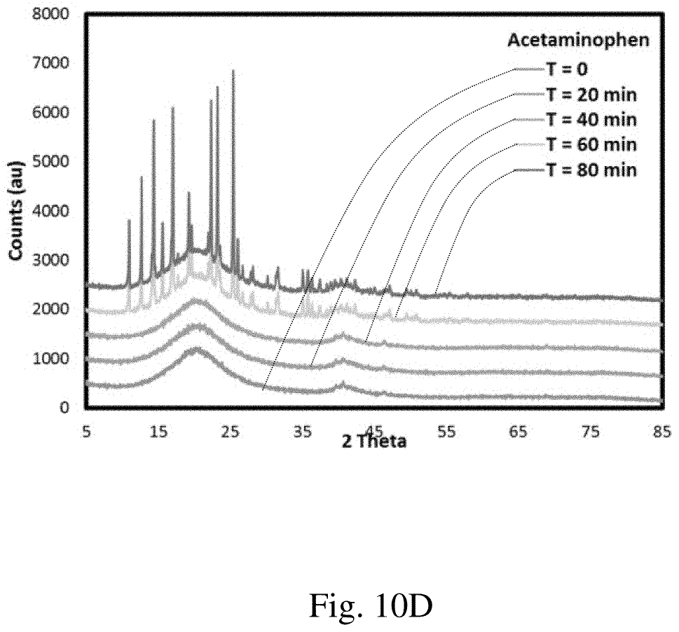

In some Zorbax® experiments, four APIs tested all crystallized within a sealed capillary after loading while being monitored in an XRPD instrument. API solutions tested in capillary tubes filled with Zorbax® functionalized media showed no crystallinity at the initial XRPD scan at 0 minutes. In the case of aspirin and nicotinamide (e.g., FIG. 10A, FIG. 10B), the second XRPD run at 20 minutes into the experiment showed crystal formation of the target molecule. A diphenhydramine hydrochloride scan at 40 minutes showed crystallinity (e.g., FIG. 10C) and an acetaminophen scan at 60 minutes showed crystallinity (e.g., FIG. 10D). Samples were held for another 20 minutes after first showing crystallinity to see that they maintained crystalline API within the sealed capillary. Non-limiting exemplary time series of XRPD scans for each of the compounds discussed are shown in FIG. 10A-FIG. 10D; each API had crystal formation within 1 hour. An artificial offset was placed in the y-axis so that each scan was visible.

Capillary Crystallization Experiment 2

Zorbax® chromatographic media with C8-like surface groups was used, to mimic the functional group interaction of alkanes, which tend to be poor solvents for the organic APIs chosen for this study. Without wishing to be bound by theory, it can be postulated that in the confined volumes of the pores of the matrix, the addition of these surface groups rendered a change in the solubility of API in solution in these nanoscale volumes, providing a driving force for nucleation and crystallization. This has in this example been applied to the crystallization of several small molecule organic compounds. Given that the parent solutions were undersaturated, when held at a fixed temperature left alone or in the presence of non-functionalized control silica media which mimicked the surface area of the Zorbax® media, the solutions did not crystallize because there was no driving force for nucleation. However, it was demonstrated in this example that the addition of the functionalized Zorbax® media induced crystallization from undersaturated parent solutions. The confined nanoscale volumes of the pores had high surface areas, and thus it was hypothesized that the contribution of the surface functionalization interaction with the solvent in this environment would be high. Without wishing to be bound by theory, the combined surface functionalization and confinement effect may have allowed for the Zorbax® media to act as an antisolvent, reducing the solubility of the APIs and causing nucleation and crystallization.

Zorbax® functionalized chromatographic media was obtained from Agilent in bulk packing form. This was porous silica functionalized with a C8-like group (n-octyldimethylsilane). The average listed grain size of the beads was 7 μm and the average pore diameter was 7 nm with a nominal surface area of 160-180 m² g⁻¹. Thermogravimetric analysis (TGA) studies were performed on the Zorbax® to determine the mass loss from C8 groups on heating and in turn calculate the density of the functional groups on the pore surface. The functional group coverage was of the order of 5 μmole m⁻². As a control, unfunctionalized controlled pore glass (CPG) was purchased from Prime Synthesis (Aston, Pa., USA). This was a fumed silica with controlled pore size of approximately 12 nm. The average grain size of this material was 100 μm.

First, capillary X-ray powder diffraction (XRPD) studies were performed to demonstrate that functionalization on the porous matrix facilitates crystallization from an undersaturated parent solution. Three systems of active pharmaceutical ingredients (APIs) were studied—diphenhydramine hydrochloride, aspirin and nicotinamide—in isopropyl alcohol as solvent. The solubility of each system at 25° C. was determined and is listed in Table 1. Next, solutions of about 85% of the solubility were prepared (actual concentrations listed in Table 1) and loaded into glass capillary columns in three sets of experiments: without any matrix, with unfunctionalized CPG, and with the functionalized Zorbax®. The capillaries were sealed to prevent solvent evaporation in order to eliminate any external influences on concentration. After a period of 1 hour to allow for any potential crystallization to occur, each capillary was mounted in a capillary spinner apparatus of the XRPD instrument and scanned. For comparison, the three dry commercial APIs were also scanned using capillary XRPD.

TABLE 1
Solubility at 25° C. and concentration of the solution loaded into
capillaries for XRPD analysis for each of the three API systems

| | Solubility at | Concentration tested with |
|---|---|---|
| API | 25° C. (mg mL⁻¹) | capillary XRPD (mg mL⁻¹) |
| Diphenhydramine | 38.0 | 30.0 |
| hydrochloride | | |
| Aspirin | 90.0 | 75.0 |
| Nicotinamide | 46.5 | 40.0 |

For each of the three systems, no crystallization was observed in the capillaries filled simply with the API solutions without any matrix. This was expected since the solutions were undersaturated and there were no potential influences on saturation. Furthermore, the capillaries with unfunctionalized CPG displayed just an amorphous background signal from the silica matrix but no presence of crystals. These results demonstrated that confinement effects from the pores alone, without any functionalization, did not induce crystallization in the undersaturated solutions studied.

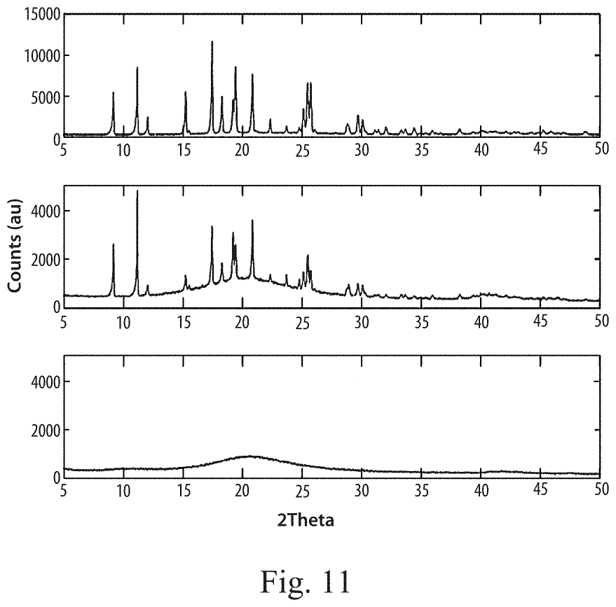

However, all three systems displayed the presence of crystals in capillaries containing the functionalized Zorbax®. The results for the first system, diphenhydramine hydrochloride are shown in FIG. 11. This demonstrated that the C8 functionalization on the Zorbax was successful in bringing about crystallization even from the undersaturated solutions.

FIG. 11 depicts XRPD scans for capillaries containing Zorbax and diphenhydramine hydrochloride systems. From top to bottom in FIG. 11, dry commercial API, 30 mg mL⁻¹ solution and 25 mg mL⁻¹ solution. No crystallization was observed in the lowest scan, validating the following hypothesis.

It was hypothesized that there exists an effective solubility of the API in the presence of the functionalized nanoporous silica, and this solubility is lower than that of the API in pure solvent. Without wishing to be bound by theory, this may be responsible for the antisolvent-like behavior of the silica matrix. Without wishing to be bound by theory, provided that there is enough matrix available for crystallization, crystals may grow within the pores until the API concentration in the mother liquor equilibrates at the effective solubility value. The “mother liquor” refers to the part of a solution that is left over after crystallization. Hence, feed solutions with API concentrations above the effective solubility would crystallize with the addition of Zorbax®, and those with concentrations below this value wouldn't crystallize. In other words, there may exist a critical minimum concentration below which no crystallization can occur, even in the presence of the functionalized Zorbax®.

In order to test this hypothesis, solutions of successively lower concentrations were loaded into capillaries containing the functionalized Zorbax® and tested for the presence of crystals. As hypothesized, for each system, crystallization failed to occur below a certain critical concentration. Results for diphenhydramine hydrochloride are shown in FIG. 11. This critical minimum concentration value lies between the lowest concentration studied which displays crystallization and the highest concentration studied which fails to display any crystallization.

This hypothesis was validated further by performing batch experiments and determining the mother liquor concentration to verify if it matched the critical value from the capillary XRPD experiments. For each system, 10 mL saturated solutions with Zorbax® added were stirred for 6 h at 25° C. The amount of Zorbax® added was 1 g for the diphenhydramine hydrochloride and the nicotinamide systems and 1.5 g for the aspirin system. The higher amount was chosen for the aspirin system to allow for sufficient matrix in view of the higher mass to be crystallized compared to the other two systems. At the end of the batch run, the solids were filtered out and the mother liquor concentration was determined via HPLC. In each system, the critical value lay in the range predicted from the capillary XRPD experiments as shown in Table 2, validating the hypothesis.

TABLE 2
Comparison of mother liquor concentration after batch
crystallization with the critical minimum concentration
range predicted (via capillary XRPD) for crystallization
to occur (conc.: concentration). In each case, the mother
liquor concentration lies within the predicted range
provided sufficient matrix is present

| | Mother liquor | Concentration range |
|---|---|---|
| API | concentration (mg mL⁻¹) | from XRPD (mg mL⁻¹) |
| Diphenhydramine | 26.4 | 25-27 |
| hydrochloride | | |
| Aspirin | 66.8 | 65-70 |
| Nicotinamide | 33.2 | 30-35 |

To examine if there was any surface crystallization in addition to crystals confined within the Zorbax® nanopores, the differential scanning calorimetry (DSC) scan of the post-crystallization Zorbax® matrix for the diphenhydramine hydrochloride system was compared with the scan of bulk API. Since the melting point of crystals decreases with size, the presence of nanocrystals can be established by a peak at a lower melting point compared to that of the bulk API. Furthermore, in the case of surface crystals in addition to the confined nanocrystals, there would be two peaks in the DSC scan-one at the lower melting point corresponding to the nanocrystals and the other at the same melting point as the bulk API corresponding to the surface crystals. The presence of a single peak at a lower melting point for the post-crystallization Zorbax® sample as shown in FIG. 9 supports that there was no observable surface crystallization and all crystals were confined to the nanopores. The significant depression in melting point indicates the nanoscale dimensions of the confined crystals.

For the diphenhydramine hydrochloride system, the single step batch yield at 25° C. starting with a saturated feed solution (concentration 38 mg mL⁻¹) and ending with a mother liquor of concentration 26.4 mg mL⁻¹ is about 30% and corresponds to a loading of 116 mg API per g Zorbax®. To improve the overall yield of the process, the mother liquor can be concentrated by evaporation and subjected to recrystallization with Zorbax® added. After each crystallization step, pure solvent preferably at a higher temperature can be used to dissolve the crystals from the Zorbax® matrix followed by filtration of the matrix and subsequent recovery of the API. This strategy can be employed in synthesis for intermediates which require a solvent switch or in the purification of an API or intermediate where an intermediate is crystallized and then dissolved in a different solvent. As a practical example, a preparative chromatography column filled with the functionalized nanoporous silica can be operated in a continuous mode for the purification process.

### Materials for Capillary Crystallization Experiment 2:

Diphenhydramine hydrochloride (DPH) was purchased from BeanTown Chemical (>99.0%). Aspirin (ASA) and nicotinamide (NIC) were purchased from Sigma Life Science (USP grade, Lot #MKBQ8444V and >99.5%, Lot #BCBF9698V, respectively). Solvents were purchased at ACS grade or higher purity from Fisher Scientific (Waltham, Mass., USA).

### Instrumentation and Conditions for Analysis for Capillary Crystallization Experiment 2:

XRPD:

XRPD was performed in a capillary setup using a PANalytical X'Pert PRO (Almelo, the Netherlands) diffractometer at 45 kV with an anode current of 40 mA. The instrument has a PW3050/60 standard resolution goniometer and a PW3373/10 Cu LFF DK241245 X-ray tube.

Samples were loaded in capillary tubes (see later section) and aligned on a goniometer capillary holder in stage capillary spinner mode with a focused point incident beam using a Cu Si focusing mirror. Settings on the incident beam path included: soller slit 0.04 rad, mask fixed 10 mm, programmable divergence slit and fixed ½° anti-scatter slit. Settings on the diffracted beam path include: soller slit 0.04 rad and ⅛° anti-scatter slit. The scan was set as a continuous scan: 2θ angle between 2° and 90°, step size 0.0167113° and a time per step of 15.240 seconds.

The saturation solubilities of aspirin, nicotinamide, and diphenhydramine hydrochloride in isopropyl alcohol were found by stirring a slurry of the API in solvent at 25° C. for several days, filtering the solution, and performing gravimetric analysis. These experiments were repeated in triplicate. The saturation solubility of diphenhydramine hydrochloride in isopropyl alcohol was found to be 38.0 mg/mL and an undersaturated solution of 30.0 mg/mL was prepared. The saturation solubility of aspirin in isopropyl alcohol was determined to be 90.0 mg/mL. An undersaturated solution of 75.0 mg/mL was prepared. Finally, the saturation solubility of nicotinamide in isopropyl alcohol was found to be 46.5 mg/mL and a solution of 40.0 mg/mL was prepared.

Glass capillary tubes were purchased from Hampton Research (Aliso Viejo, Calif.). The tubes were made of glass #50, with a 1.0 mm OD, wall thickness of 0.01 mm and length of 80 mm. The capillary tubes were packed with Zorbax® or CPG material by scooping powder into the tip, placing the capillary in a larger diameter glass pipette, and tapping to allow gravity to act on the powder to settle. Once packed, the tubes were suctioned at the open end with a 1-mL plastic syringe, using a small piece of rubber tubing to adapt the fit. The filled capillary was then immediately dipped into the undersaturated API solutions to allow the solution to fill the tube. Molten wax was used to plug the end of the capillary, and the tip was then heated in a Bunsen burner flame to seal. Additional capillaries were filled with the API solution and dry commercial API alone.

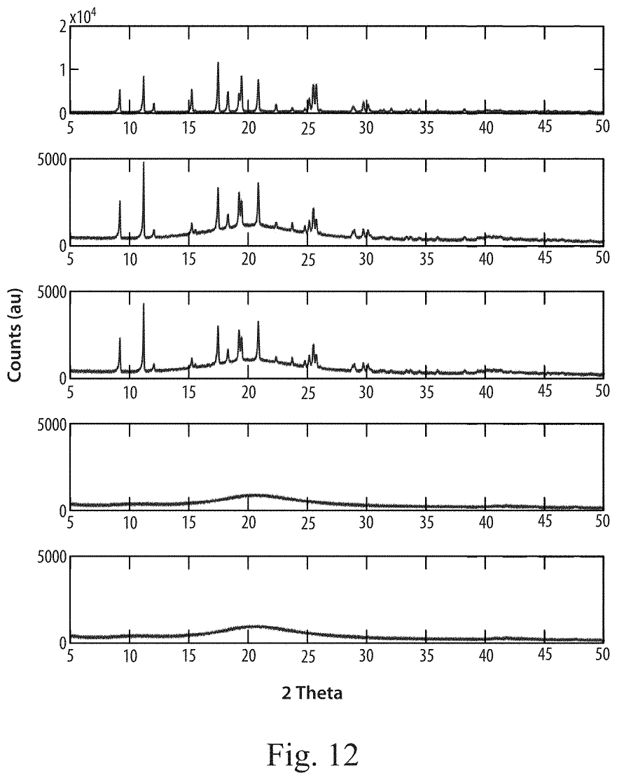

To check the hypothesis that there exists a critical minimum undersaturation below which the Zorbax® cannot induce crystallization, solutions of successively decreasing concentrations were tested with the capillary method. The results supported the hypothesis and are shown in FIG. 12 to FIG. 14.

FIG. 12 shows XRPD scans for diphenhydramine systems. From top to bottom of FIG. 12: commercial API, 30 mg/mL, 27 mg/mL, 25 mg/mL and 23 mg/mL.

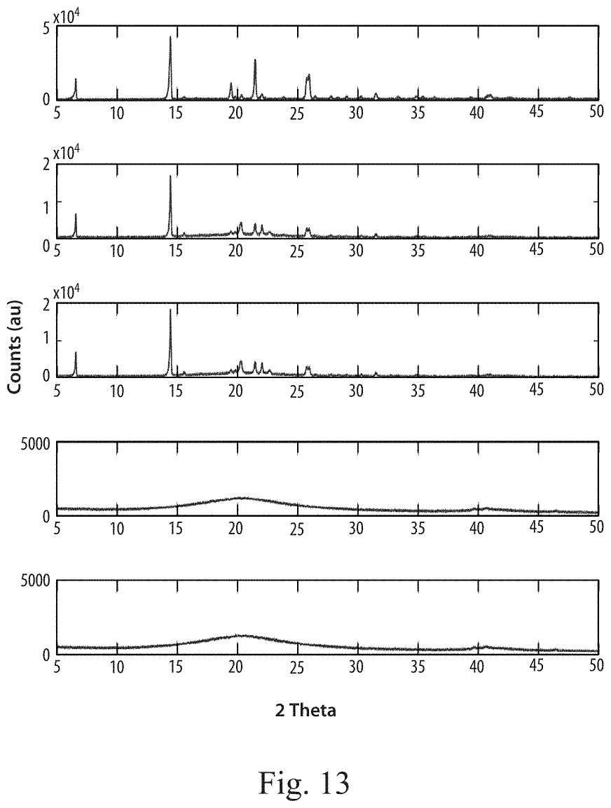

FIG. 13 shows XRPD scans for Aspirin systems. From top to bottom of FIG. 13: commercial API, 75 mg/mL, 70 mg/mL, 65 mg/mL and 60 mg/mL

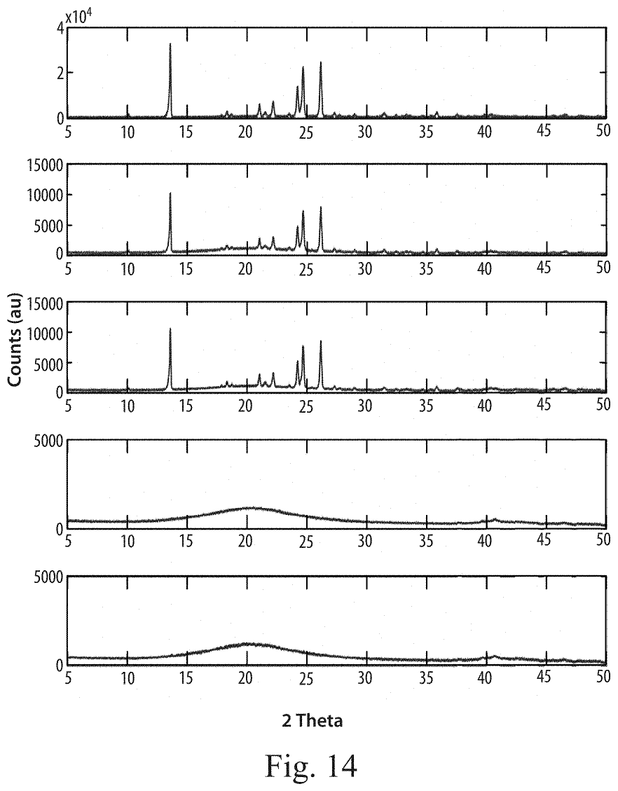

FIG. 14 shows XRPD scans for Nicotinamide systems. From top to bottom of FIG. 14: commercial API, 40 mg/mL, 35 mg/mL, 30 mg/mL and 25 mg/mL.

TGA:

Thermogravimetric analysis (TGA) was performed on a Q500 instrument from TA instruments (Newcastle, Del., USA) connected with a nitrogen gas cylinder to maintain a flow rate of 25 mL/min to maintain an inert gas environment in the sample chamber. Between 5 and 10 mg of sample were loaded on platinum sample pans from TA Instruments. The samples were allowed to equilibrate at 30° C. and then heated at 10° C./min to 600° C.

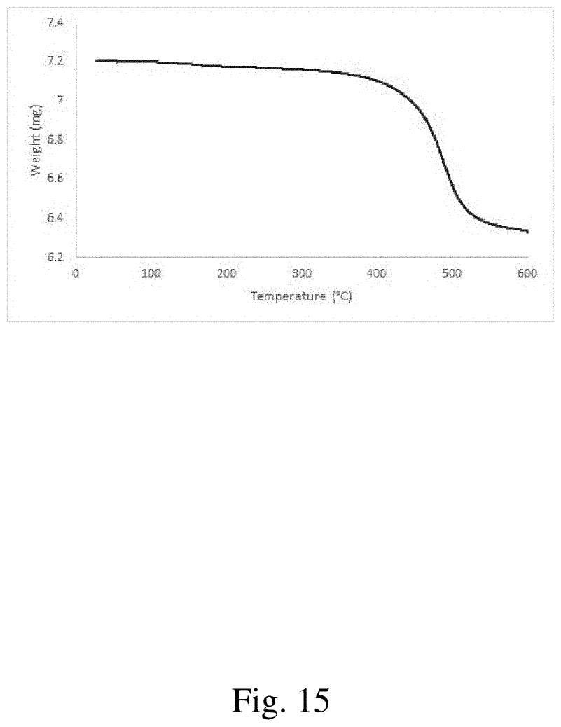

The extent of functionalization on the commercial Zorbax® matrix was studied using TGA. The weight as a function of temperature for one run is shown in FIG. 15. Without wishing to be bound by theory, the mass loss may correspond to C8 functionalization. The average mass loss was 12.23% of the functionalized matrix, which may translate to the C8 groups amounting to 139.4 mg/g silica matrix. The commercial literature states that the groups are n-octyldimethylsilane (having a molecular weight of 172.4 g/mol) and the nominal surface area of the matrix is 160-180 m²/g. Thus, the density of groups can be calculated to be 2.86×10¹⁸ groups/m² pore surface area corresponding to about 5 μmole/m². This value can be used for comparison of functionalization efficiency with any functionalization performed in-house in the future.

FIG. 15 shows a DSC scan for functionalized Zorbax showing mass loss as a function of temperature.

DSC:

A Q2000 instrument from TA instruments was used for the differential scanning calorimetry (DSC) analysis. Inert atmosphere environment was maintained in the sample chamber using a nitrogen gas cylinder set to a flow rate of 50 mL/min. An extra refrigerated cooling system (RCS 40, TA Instruments) was used to widen the available temperature range to between −40° C. and 400° C. Tzero pans and lids were used with ˜5 mg of sample. A heating rate of 10° C./min was applied and the samples were scanned from −20° C. to 200° C.

Without wishing to be bound by theory, the relationship between a crystal of characteristic dimension d and its melting point temperature Tₘ(d) may be dictated by a modified Gibbs-Thompson relationship given by:

Tₘ−Tₘ(d)=[4(γ_{solid-substrate}−γ_{liquid-substrate})MTₘ]/(dΔH_{fus}ρ_{solid}) Equation 1

Where Tₘ is the bulk melting point temperature, M is the molecular mass, ρ_{solid} is the density of the solid bulk crystals, ΔH_{fus} is its molar enthalpy of fusion and γ_{solid-substrate} and γ_{liquid-substrate} are the surface free energies of the interface between the substrate and the solid and the liquid phases respectively.

A linear relationship has been found exist between Tₘ(d) and 1/d for fenofibrate whereby the decrease in the surface free energies (in the numerator of Equation 1) with crystal size is offset by the decrease in the enthalpy of fusion (in the denominator of Equation 1). Based on these results, the scale of melting point depression, seen in the current work, is consistent with crystal dimensions in the nanometer scale (recall that the average pore size of the Zorbax® matrix is 7 nm).

HPLC:

An Agilent 1100 instrument equipped with a UV diode array detector was used for high performance liquid chromatography (HPLC) analysis of the post-crystallization mother liquor. The assay methods were obtained from US Pharmacopeia version 40 and are listed below:

Diphenhydramine Hydrochloride:

The column was Ascentis® ES-Cyano column (Sigma-Aldrich) of dimensions 150 mm×4.6 mm i.d. packed with 5 micron particles of 12 nm pore size. An isocratic method with a mobile phase of 50:50:0.5 by volume of acetonitrile, water and trimethylamine adjusted with glacial acetic acid to a pH of 6.5 at a flow rate of 1 mL/min was used. The detection wavelength was set to 254 nm.

Aspirin:

The column was YMC-Pack ODS-A column (YMC America Inc.) of dimensions 150 mm×4.6 mm i.d. packed with 3 micron particles of 12 nm pore size. An isocratic method with a mobile phase of 69:28:3 by volume of water, methanol and glacial acetic acid at a flow rate of 1 mL/min was used. The detection wavelength was set to 280 nm.

Nicotinamide:

The column was YMC-Pack ODS-A column (YMC America Inc.) of dimensions 150 mm×4.6 mm i.d. packed with 3 micron particles of 12 nm pore size. An isocratic method with a mobile phase of 20:20:60 by volume of methanol, acetonitrile and water at a flow rate of 1 mL/min was used. The detection wavelength was set to 230 nm.

This work successfully demonstrates that a surface-functionalized nanoporous media may be used to provide surface functionality in confined nano-volumes of the pores which results in a reduced solubility within the confined volume resulting in the formation of nanocrystals within the pores, e.g., from an undersaturated parent solution.

Commercial Entities that May be Interested in this Disclosure

Embodiments of this disclosure may be highly applicable in the pharmaceutical industry, as fine control over crystallization of commodity APIs is of interest and these APIs may be both heat- and solvent-sensitive. Embodiments of this disclosure may also be applicable to chemical products crystallization. Embodiments of this disclosure may also be applicable in the crystallization of proteins and macromolecules. Another relevant application of this principle may be in use for a purification technique, where an impure API or intermediate solution may be flowed through a column with chosen antisolvent-like functionality for the API alone, to selectively crystallize API within the pores of the matrix while enriching flowthrough in impurity. An eluent solution could be then flowed over the column to recapture a purer API solution.

### EQUIVALENTS

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall be interpreted as indicating exclusive alternatives (i.e. “one or the other but not both”) only when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of forming crystals, the method comprising:
obtaining a solution of a target molecule, wherein the solution is at an undersaturated state with respect to the target molecule; and
exposing the solution to a functionalized nanoporous matrix under conditions that promote crystal formation of the target molecule.

2. The method of claim 1, wherein the matrix is functionalized with an antisolvent group.

3. A method of forming crystals, the method comprising:
obtaining a solution of a target molecule; and
exposing the solution to an antisolvent-functionalized nanoporous matrix, under conditions that promote crystal formation of the target molecule.

4. The method of claim 1, wherein conditions that promote crystal formation of the target molecule include isothermal conditions.

5. The method of claim 1, wherein conditions that promote crystal formation of the target molecule include a temperature, of an environment external to the solution and the matrix, of between or equal to a melting temperature and a boiling temperature of the solution at 1 atm pressure.

6. The method of claim 1, wherein conditions that promote crystal formation of the target molecule include a temperature, of an environment external to the solution and the matrix, of between or equal to a melting temperature and a boiling temperature of the target molecule at 1 atm pressure.

7. The method of claim 1, wherein conditions that promote crystal formation of the target molecule include a temperature, of an environment external to the solution and the matrix, of between or equal to 20° C. and 30° C. and a pressure of the external environment of 1 atm.

8. The method of claim 1, comprising exposing the solution to the matrix under conditions that promote crystal formation of the target molecule within at least one nanopore in the matrix.

9. The method of claim 1, further comprising preparing the solution.

10. The method of claim 1, further comprising functionalizing a surface of the matrix.

11. The method of claim 1, further comprising eluting at least a portion of one or more crystals from the matrix.

12. The method of claim 1, wherein exposing comprises flowing the solution through at least a portion of the matrix.

13. A system for forming crystals, the system comprising:
a first container comprising a functionalized nanoporous matrix; and
a solution of a target molecule, wherein:
(i) the solution is at an undersaturated state with respect to the target molecule; and/or
(ii) the functionalized nanoporous matrix is an antisolvent-functionalized nanoporous matrix configured and arranged to promote crystallization of the target molecule from the solution.

14. (canceled)

15. The system of claim 13, wherein the matrix is configured as a packed bed in a portion of the container.

16. The system of claim 13, wherein the matrix is configured as a monolithic body in a portion of the container.

17. The method of claim 1, wherein the matrix has a specific surface area of between or equal to 100 m²/gram and 500 m²/gram.

18. The method of claim 1, wherein the matrix has an average pore size of between or equal to 1 nm and 100 nm.

19. The method of claim 1, wherein the target molecule is an active pharmaceutical ingredient (API).

20. The method of claim 1, wherein the target molecule is a protein.

21. The system of claim 13, wherein the system further comprises a second container comprising the solution.
